(19) United States
(12) Patent Application Publication
(10) Pub. No.: US 2021/0215704 A1
(43) Pub. Date: Jul. 15, 2021

(54) LABELED SILICA-COATED GOLD NANORODS AND A METHOD FOR PRODUCING THE SAME

(71) Applicant: UNIVERSITY OF YAMANASHI, Kofu-shi (JP)

(72) Inventors: Hideyuki Shinmori, Kofu-shi (JP); Kei Yamazaki, Kofu-shi (JP); Akira Shinohara, Kofu-shi (JP)

(73) Assignee: UNIVERSITY OF YAMANASHI, Kofu-shi (JP)

(21) Appl. No.: 17/144,877

(22) Filed: Jan. 8, 2021

(30) Foreign Application Priority Data
Jan. 9, 2020 (JP) 2020-002077
Dec. 18, 2020 (JP) 2020-209844

(51) Int. Cl. G01N 33/58; A61K 41/00; A61K 47/69; G01N 21/64

(52) U.S. Cl. CPC G01N 33/582; A61K 41/0052; G01N 2021/6439; G01N 21/6428; A61K 47/6923

(57) ABSTRACT

An object of the present invention is to provide fluorescently labeled silica-coated gold nanorods that are safe for administration to living bodies, stable to temperature rise and external environment, and easy to manufacture. The present invention is a labeled silica-coated gold nanorod, including a gold nanorod, a silica layer covering the gold nanorod, spacers bonded to the silica layer, and labeled materials, in which the labeled material is chemically bonded to the spacer. The present invention also provides a method for producing a labeled silica-coated gold nanorod, including an introduction step and a binding step, in which in the introduction step, spacers are introduced on a silica layer of a silica-coated gold nanorod and in the binding step, a labeled material is chemically bound to the spacer.

### TECHNICAL FIELD

The present invention relates to gold nanoparticles, in particular, silica-coated gold nanorods bonded with labeled materials.

### BACKGROUND ART

Gold nanoparticles are attracting attention for applications as nanomaterials such as bio-imaging, contrast and labeling agents, and biosensors, and even as photothermal nanotherapeutics due to their characteristic optical properties in the visible light range. In fact, the gold nanoparticles are used as colorants in commonly available pregnancy test kits and in the simple diagnosis of influenza used in hospitals.

Among these gold nanoparticles, gold nanorods, which are rod-shaped gold nanoparticles, are useful in bioscience because their light absorption and light scattering wavelengths can be extended to the near-infrared region (600 to 900 nm), which is called the “biological window” for tissue permeability. However, the gold nanorods have problems about shape stabilization in the nano-size range and quenching phenomena due to light energy transfer near the interface thereof, which makes it difficult to apply as a higher sensitive luminescent agent.

For luminescence important for sensitive bio-imaging and other applications, it has been demonstrated that the fluorescence intensity depends on the distance/spacer length between the core metal and the fluorescent part. That is, as the spacer length decreases, the fluorescence intensity decreases. For this reason, polymers and DNA have been used as spacers to adjust the luminescence (Non-Patent Document 1: Appl. Phys. Lett., 2009, 94, 063111; J. Am. Chem. Soc., 2006, 128, 5462-5467).

In addition, contrast agents loaded with (ICG) on a porous silica layer with which gold nanorods are coated to obtain X-ray CT and NIR fluorescence imaging images have been used in cancer testing (Non-Patent Document 2: Optics Express, 2011 Vol. 19, No. 18, 17030-17039).

Patent Document 1 (JP2016-216547A) discloses the invention of core-shell gold nanoparticles that contain phosphors and use the “surface plasmon effect” to enhance the fluorescence generated from the phosphors in display devices for color displays and light sources that emit colored light.

### SUMMARY OF INVENTION

### Technical Problem

However, the spacers in Non-Patent Document 1 are organic materials and have a a problem of lacking flexibility and stability.

Therefore, the inventors attempted to prepare silica-coated gold nanorods using a silane coupling agent as a spacer. However, as shown in the comparative examples discussed below, the absorption spectrum showed a clear decrease in the absorption band due to the lack of sample dispersion, and its FE-SEM image revealed that no silica coating was made.

Next, the inventors attempted to perform silica coating and fluorescence labeling of gold nanorods using a mixture of tetraethoxysilane and a silane coupling agent. However, it was found that the fluorescence intensity of the fluorescently labeled silica-coated gold nanorods produced by this production method was reduced.

In addition, the contrast agents composed of gold nanorods disclosed in Non-Patent Document 2 lack stability against temperature rise and external environment because the fluorescent material is not chemically bonded but physically trapped only. Furthermore, the fluorophores disclosed in Patent Document 1 are difficult to manufacture because the distance between the metal nanostructures and the fluorophores must be short and strictly maintained to take advantage of the surface plasmon effect, and the safety of the fluorophores when administered to the living bodies has not been considered.

Finally, the inventors revealed that fluorescence-labeled silica-coated gold nanorods with no decrease in fluorescence intensity could be produced by introducing a silica coupling agent into the silica layer of gold nanorod silica-coated with tetraalkoxysilane and binding a fluorescent material to the silane coupling agent, and then the present invention has been completed.

An object of the present invention is to provide fluorescently labeled silica-coated gold nanorods that are safe for administration to living bodies, stable to temperature rise and external environment, and easy to manufacture.

### Solution to Problem

The present invention is a labeled silica-coated gold nanorod, including a gold nanorod, a silica layer covering the gold nanorod, spacers bonded to the silica layer, and labeled materials, in which the labeled material is chemically bonded to the spacer.

This can provide the labeled silica-coated gold nanorod that do not reduce the fluorescence intensity of the fluorescent material to the extent that it does not interfere with practical use.

The thickness of the silica layer may be 15 nm or more. The thickness of the silica layer keeps the distance between the labeled material and the gold nanorod, so the fluorescence intensity is not reduced.

The spacer may be derived from a silane coupling agent including a Si atom and four functional groups directly or indirectly connected to the Si atom. The four functional groups may have at least one inorganic functional group and at least one organic functional group.

The organic functional group may be at least one selected from the group consisting of a vinyl group, an epoxy group, a styryl group, a methacrylic group, an acrylic group, an amino group, an ureide group, an isocyanate group, an isocyanurate group, and a mercapto group.

The organic functional group may be indirectly connected to the Si atom via an alkyl group having 1 to 5 carbons, an alkoxy group having 1 to 5 carbons, a phenyl group, a heterocyclic group, or a fused ring group.

The spacer may be vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, P-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethylbutylidene)propylamine, N-phenyl-3-aminopropyl trimethoxysilane, 3-ureidopropyltrialkoxysilane, 3-isocyanatepropyltriethoxysilane, tris-[(trimethoxysilyl)propyl]isocyanurate, (3-mercaptopropyl)methyldimethoxysilane, or 3-mercaptopropyltrimethoxysilane.

The present invention also provides a method for producing a labeled silica-coated gold nanorod, including an introduction step and a binding step, in which in the introduction step, spacers are introduced on a silica layer of a silica-coated gold nanorod and in the binding step, a labeled material is bound to the spacer.

This can produce the labeled silica-coated gold nanorod that do not reduce the fluorescence intensity of the fluorescent material to the extent that it does not interfere with practical applications.

The thickness of the silica layer may be 15 nm or more. The thickness of the silica layer keeps the distance between the labeled material and the gold nanorod, so the fluorescence intensity is not reduced.

### BRIEF DESCRIPTION OF DRAWINGS

FIG. 1 shows a synthesis scheme of a hexadecyltrimethylammonium bromide (CTAB)-protected AuNR using a seed-mediated method.

FIG. 2 shows an absorption spectrum of the AuNR.

FIG. 3 shows a field emission scanning electron microscope (FE-SEM) photograph of the AuNR.

FIG. 4 shows particle size distribution of the AuNR calculated from the FE-SEM photograph.

FIG. 5 shows a preparation scheme of silica-coated AuNR (AuNR@TEOS) by using tetraethoxysilane (TEOS).

FIG. 6 shows absorption spectra of the AuNR and AuNR@TEOS.

FIG. 7 shows Zeta potential of the AuNR and AuNR@TEOS.

FIG. 8 shows Fourier transform infrared spectroscopy (FT-IR) spectra of the AuNR and AuNR@TEOS.

FIG. 9 shows a FE-SEM photograph of the AuNRs@TEOS.

FIG. 10 shows silica layer distribution of the AuNRs@TEOS calculated from the FE-SEM photograph.

FIG. 11 shows absorption spectra of the AuNR, AuNR@TEOS and AuNRs@APTES.

FIG. 12 shows FE-SEM photograph of the AuNRs@APTES.

FIG. 13 shows a preparation scheme of the AuNR@TEOS-APTES by introducing 3-aminopropyltriethoxysilane (APTES) (—NH₂ group) to the AuNR@TEOS.

FIG. 14 shows absorption spectra of the AuNR, AuNR@TEOS and AuNR@TEOS-APTES.

FIG. 15 shows Zeta potential of the AuNR, AuNR@TEOS and AuNR@TEOS-APTES.

FIG. 16 shows FT-IR spectra of the AuNR, AuNR@TEOS and AuNR@TEOS-APTES.

FIG. 17 shows FE-SEM photograph of the AuNRs@TEOS-APTES.

FIG. 18 shows silica layer distribution of the AuNR@TEOS-APTES calculated from the FE-SEM photograph.

FIG. 19 shows a preparation scheme of AuNR@TEOS-APTES-Dansyl by modifying the AuNR@TEOS-APTES with a Dansyl group using Dansyl Chloride.

FIG. 20 shows absorption spectra of the AuNR@TEOS-APTES, AuNR@TEOS-APTES-Dansyl and Dansylated hexylamine.

FIG. 21 shows an enlarged graph of the graph shown in FIG. 20.

FIG. 22 shows a spectrum representing the difference in absorption spectra between the AuNR@TEOS-APTES and AuNRs@TEOS-APTES-Dansyl (i.e., the difference before and after the Dansyl group modification).

FIG. 23 shows FT-IR spectra of the AuNR, AuNR@TEOS, AuNR@TEOS-APTES and AuNR@TEOS-APTES-Dansyl.

FIG. 24 shows Zeta potential of the AuNR, AuNR@TEOS, AuNR@TEOS-APTES and AuNR@TEOS-APTES-Dansyl.

FIG. 25 shows FE-SEM photograph of the AuNR@TEOS-APTES-Dansyl.

FIG. 26 shows silica layer distribution of the AuNR@TEOS-APTES-Dansyl calculated from the FE-SEM photograph.

FIG. 27 shows the fluorescence spectra of the AuNR, AuNR@TEOS, AuNR@TEOS-APTES and AuNR@TEOS-APTES-Dansyl.

FIG. 28 shows photographs of UV (365 nm) irradiation of the AuNR, AuNR@TEOS, AuNR@TEOS-APTES and AuNR@TEOS-APTES-Dansyl in each vial.

FIG. 29 shows fluorescence spectra of the AuNR@TEOS-APTES-Dansyl and the standard, Quinine Sulfate Dihydrate at the first time.

FIG. 30 shows fluorescence spectra of the AuNR@TEOS-APTES-Dansyl and the standard, Quinine Sulfate Dihydrate at the second time.

FIG. 31 shows fluorescence spectra of the AuNR@TEOS-APTES-Dansyl and the standard, Quinine Sulfate Dihydrate at the third time.

### DESCRIPTION OF EMBODIMENTS

### Definition

For convenience, certain terms employed in the context of the present disclosure are collected here. Unless defined otherwise, all technical and scientific terms used herein have the same meaning as commonly understood by one of the ordinary skilled in the art to which this invention belongs. The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are described as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art.

Hereinafter, embodiments of the present invention are illustrated in detail. The following embodiments are illustrative only and do not limit the scope of the present invention. In order to avoid redundancy, explanation for similar contents is not repeated.

### Embodiment 1

A labeled silica-coated gold nanorod of the present embodiment includes a gold nanorod, a silica layer covering the gold nanorod, spacers bonded to the silica layer, and labeled materials, in which the labeled material is chemically bonded to the spacer.

The labeled silica-coated gold nanorod of the present embodiment includes the gold nanorod. The purity of the gold nanorod used in the present embodiment may be 75, 80, 85, 90, 95, 98, 99, 99.9 or 99.99% or more, or may be within a range between any two of the values illustrated herein. The long axis of the gold nanorod used in the present embodiment may be 3, 10, 18, 32, 100, 180, 280, 400, 540 or 800 nm, or may be within a range between any two of the values illustrated herein. The short axis of the gold nanorod used in the present embodiment may be 2, 5, 6, 8, 20, 30, 40, 50, 60 or 80 nm, or may be within a range between any two of the values illustrated herein. The aspect ratio (long axis/short axis) of the gold nanorod used in the present embodiment may be 1.5, 2, 3, 4, 5, 6, 7, 8, 9 or 10, or may be within a range between any two of the values illustrated herein. The long and short axes of the gold nanorod can be measured from photographs taken by a scanning electron microscope.

In the present embodiment, the average particle size of the gold nanorods may be 20, 21, 22, 23, 24, 25, 26, 27, 28, 29, or 30 nm or may be within a range between any two of the values illustrated herein. The average particle size of the gold nanorods refers to the diameter of the particle in 50% of the integrated value in the particle size distribution obtained by measuring the projected area circle equivalent diameter of the particles from 200 particles randomly selected using photographs taken by a scanning electron microscope. The average particle size of the gold nanorods may be calculated using a dynamic light scattering (DLS) particle size distribution instrument.

In the present embodiment, the silica-coated gold nanorod is covered with a silica layer. The thickness of the silica layer may be 15, 20, 25, 30, 35, 40 or 45 nm or may be within a range between any two of the values illustrated herein. The thickness of the silica layer can be measured from photographs taken by a scanning electron microscope. The coverage of the silica layer to the gold nanorod may be 60, 70, 80, 90, 95, 98, 99, 99.9, 99.99% or more, or may be within a range between any two of the values illustrated herein. The coverage of the silica layer can be calculated from the length of the portion of the gold nanorod coated with silica per full circumference of the gold nanorod, measured from a photograph taken by a scanning electron microscope. The gold nanorod is covered with the silica layer to stabilize its shape in the nanosize region. The silica layer covering the gold nanorod can be produced by coating the surface of the gold nanorod with silica using alkoxysilane (e.g., methyltrimethoxysilane, dimethyldimethoxysilane, phenyltrimethoxysilane, dimethoxydiphenylsilane, n-propyltrimethoxysilane, hexyltrimethoxysilane, decyltrimethoxysilane, 1,6-bis(trimethoxysilyl) hexane, trifluoropropyltrimethoxysilane, tetraethoxysilane, methyltriethoxysilane, dimethyldiethoxysilane, phenyltriethoxysilane, n-propyltriethoxysilane, hexyltriethoxysilane, octyltriethoxysilane).

In the present embodiment, the spacers are bonded to the silica layer. The spacer is derived from a silane coupling agent, and the silane coupling agent has an Si atom and four functional groups directly or indirectly connected to the Si atom, and the four functional groups have at least one inorganic functional group and at least one organic functional group. In the present embodiment, the molar ratio of the silica-coated gold nanorod:the spacers introduced on the silica-coated gold nanorod may be 1:2 to 20 (e.g., 2, 3, 4, 5, 6, 7, 8, 9, 10, 11, 12, 13, 14, 15, 16, 17, 18, 19 or 20, or within a range between any two of the values illustrated herein).

The organic functional group may be selected from the group consisting of, but not limited to, a vinyl group, an epoxy group, a styryl group, a methacrylic group, an acrylic group, an amino group, an ureide group, an isocyanate group, an isocyanurate group, and a mercapto group. The organic functional group may be indirectly connected to the Si atom via an alkyl group having 1 to 5 carbons, an alkoxy group having 1 to 5 carbons, a phenyl group, a heterocyclic group or a fused ring group.

The inorganic functional group is a group with which silanol produced by hydrolysis is hydrogen-bonded to a hydroxyl group of an inorganic material (e.g., glass and silica), preferably an alkoxy group, more preferably a methoxy or ethoxy group. The inorganic functional group may be indirectly connected to the Si atom via an alkyl group having 1 to 5 carbons, an alkoxy group having 1 to 5 carbons, a phenyl group, a heterocyclic group or a fused ring group.

In the present embodiment, the four functional groups may have an alkyl group (methyl, ethyl, propyl or isopropyl group) having 1 to 3 carbons in addition to the inorganic and organic functional groups. For example, the four functional groups may have: three inorganic functional groups and one organic functional group; two inorganic functional groups and two organic functional groups; or two inorganic functional groups, one organic functional group, and one alkyl group having 1 to 3 carbons.

The spacer may be vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, P-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethylbutylidene)propylamine, N-phenyl-3-aminopropyl trimethoxysilane, 3-ureidopropyltrialkoxysilane, 3-isocyanatepropyltriethoxysilane, tris-[(trimethoxysilyl)propyl]isocyanurate, (3-mercaptopropyl)methyldimethoxysilane, or 3-mercaptopropyltrimethoxysilane.

In the present embodiment, the spacer is chemically bound to the labeled material such as fluorescent material, luminescent material and radioactive material, resulting in being strong and stable. Therefore, the bond does not dissociate from each other due to the temperature rising or the external environment. The labeled material include, but are not limited to, an enzyme such as a peroxidase and alkaline phosphatase, radioactive material such as ¹²⁵I, ¹³¹I, ³⁵S, and ³H, fluorescein, rhodamine, dansyl, pyrene, anthraniloyl, nitrobenzoxadiazole, cyanine dye such as Cy3, and Cy5, phycoerythrins, tetramethylrhodamine, a fluorescent protein such as a green fluorescent protein from Aequorea victoria, a fluorescent protein from hermatypic coral, and a fruit fluorescent proteins a fluorescent material such as a near-infrared fluorescent material, a luminescent material such as luciferase, luciferin, and egolin and a nanoparticle such as a quantum dot. The labeled material may be a biotin-avidin (or -streptavidin) complex containing avidin or streptavidin labeled with the labeled material or a succinimidyl ester compound in which the labeled material is bound to a succinimide. The labeled material of the present embodiment and the organic functional group bound to the labeled material may be modified as appropriate for the intended use of the labeled silica-coated gold nanorods. In the present embodiment, the molar ratio of the silica-coated gold nanorods into which the spacers are introduced: the labeled materials introduced into the spacers may be 1:2 to 20 (e.g., 2, 3, 4, 5, 6, 7, 8, 9, 10, 11, 12, 13, 14, 15, 16, 17, 18, 19 or 20 or within a range between any two of the numbers illustrated herein) when the spacer has one organic functional group, and may be 1:2 to 40 (e.g., 2, 3, 4, 5, 6, 7, 8, 9, 10, 11, 12, 13, 14, 15, 16, 17, 18, 19, 20, 22, 24, 26, 28, 30, 32, 34, 36, 36, 38, or 40, or within a range between any two of the numbers illustrated herein) when the spacer has two organic functional groups.

In the present embodiment, the gold nanorod is covered with a silica layer having a thickness of at least 15 nm, and the gold nanorod and the labeled material are separated by at least 15 nm because the labeled material is chemically bonded to the silica layer via the spacer. Therefore, quenching phenomenon due to, for example, light energy transfer near the interface does not occur, resulting that a stable and highly sensitive luminescent agent can be realized.

### Embodiment 2

In accordance with the present embodiment, a method for producing a labeled silica-coated gold nanorod includes an introduction step and a binding step, in which in the introduction step, spacers are introduced on a silica layer of a silica-coated gold nanorod and in the binding step, a labeled material is bound to the spacer.

In the present embodiment, introduction conditions of the introduction step can be changed to depending on the type of the spacer to be introduced. The introduction conditions can be based on known methods. For example, when 3-aminopropyltriethoxysilane (APTES) is used as a spacer, the introduction step has a first mixing step and a second mixing step. In the first mixing step, a NaOH solution and a MeOH solution in which the silica-coated gold nanorods are dispersed are mixed while stirring. In the second mixing step, the mixed solution obtained by the first mixing step and the MeOH solution in which APTES is dissolved are mixed while stirring. The concentration ratio of the silica-coated gold nanorods:NaOH in the first mixing step may be 1:0.8 to 1.5 (e.g., 0.8, 1.0, 1.1, 1.2, 1.3, or 1.4, or 1.5, or within a range between any two of the values illustrated herein). The concentration ratio of silica-coated gold nanorods:APTES in the second mixing step may be 1:3 to 6 (e.g., 3, 3.5, 4, 4.5, 5, 5.5, or 6, or within a range between any two of the values illustrated herein). The introduction step may have a heating and stirring step. In the heating and stirring step, the mixed solution is stirred for 1, 2, 3, 4, 5, 6, 7, 8, 9, or 10 hours or a range between any two of the values illustrated herein after the second mixing step under temperature conditions of 40° C. to 60° C.

In the first mixing step, the NaOH solution may be added to the MeOH solution in which the silica-coated gold nanorods are dispersed in two to four installments every 20 to 40 minutes. In the first mixing step, the MeOH solution in which the silica-coated gold nanorods are dispersed may be 20, 22, 24, 26, 28, or 30% MeOH solution or may be MeOH solution in a range between any two of the values illustrated herein. The stirring time in the second mixing step may be 1, 2, 3, 4, 5, 6, 7, 8, 9 or 10 minutes or may be in a range between any two of the values illustrated herein.

In the present embodiment, binding conditions of the binding step can be changed depending on the type of the labeled material to be chemically bonded and the type of the organic functional group of the spacer. The chemical bonding conditions can be based on known methods. For example, when a spacer having an amino group as an organic functional group and a dansyl (dansyl group) as a labeled material are used, the binding step includes a third mixing step and a heating reflux step. In the third mixing step, triethylamine and a dried CH₂Cl₂ solution in which the silica-coated gold nanorods to which the spacers are introduced are dispersed are mixed while stirring under a nitrogen atmosphere. In heating reflux step, the mixed solution obtained by the third mixing step and the Dried CH₂Cl₂ solution in which dansyl chloride is dissolved are mixed and heated to reflux for 4, 5, 6, 7, 8, 9, 10, 11, or 12 hours or a range between any two of the values illustrated here. The heating reflux is performed under temperature conditions of 35° C. to 45° C. The concentration ratio of the silica-coated gold nanorods to which the spacer is introduced: the triethylamine in the third mixing step may be 1:0.8 to 5 (e.g., 0.8, 1.0, 1.1, 1.2, 1.3, 1.4, 1.5, 2.0, 3.0, 4.0, or 5.0, or within a range between any two of the values illustrated herein). The concentration ratio of silica-coated gold nanorods to which the spacers are introduced dansyl chloride in the heating reflux step may be 1:20 to 40 (e.g., 20, 22, 24, 26, 28, 30, 32, 34, 36, 38, or 40, or within a range between any two of the values illustrated herein).

The method for producing the labeled silica-coated gold nanorod according to the present embodiment may include a coating step, in which in the coating step, the gold nanorod is covered with silica, and may include a manufacturing step and a coating step, in which in the manufacturing step, the gold nanorod is produced and in the coating step, the gold nanorod is covered with silica.

### Manufacturing Step for Producing Gold Nanorods

The manufacturing step for producing the gold nanorods includes, for example, a seed solution preparation step, a primary growth solution preparation step, and a secondary growth Solution preparation step.

### Seed Solution Preparation Step

The seed solution preparation step includes a first seed solution preparation step, a second seed solution preparation step, a third seed solution preparation step, a fourth seed solution preparation step and a fifth seed solution preparation step, in which: in the first seed solution preparation step, a hexadecyltrimethylammonium bromide (CTAB) solution is provided; in the second seed solution preparation step, a potassium bromide solution is added to the CTAB solution; in the third seed solution preparation step, a gold(III) chloride solution is added to the mixed solution obtained in the second seed solution preparation step; in the fourth seed solution preparation step the mixed solution obtained in the third seed solution preparation step is mixed with a sodium borohydride solution while stirring; and in the fifth seed solution preparation step, the mixed solution obtained in the fourth seed solution preparation step allows to stand for 30, 45, 60, 75, or 80 minutes or a range between any two of the values illustrated herein at a temperature condition of 20 to 40° C.

The seed solution preparation step may have a seed solution preparation leaving step, in which, after the fourth seed solution preparation step, the mixed solution obtained in the fourth seed solution preparation step allows to stand for 1, 2, 3, 4, 5, 6, 7, 8, 9 or 10 minutes or a range between any two of the values illustrated herein.

In the seed solution preparation step, the concentration ratio of CTAB:potassium bromide may be 1:0.05 to 0.15 (e.g., 0.05, 0.06, 0.07, 0.08, 0.09, 0.10, 0.11, 0.12, 0.13, 0.14, or 0.15, or within a range between any two of the values illustrated herein). In the seed solution preparation step, the concentration ratio of CTAB:gold(III) chloride may be 1:0.001 to 0.003 (e.g., 0.0015, 0.0016, 0.0017, 0.0018, 0.0019, 0.0020, 0.0021, 0.0022, 0.0023, 0.0024, 0.0025, 0.0026, 0.0027, 0.0028, 0.0029, or 0.0030, and within a range between any two of the values illustrated herein). In the seed solution preparation step, the concentration ratio of CTAB:sodium borohydride may be 1:0.001 to 0.010 (e.g., 0.001, 0.002, 0.003, 0.004, 0.005, 0.006, 0.007, 0.008, 0.009, or 0.010, or within a range between any two of the values illustrated herein).

### Primary Growth Solution Preparation Step

The primary growth solution preparation step includes a first step of the primary growth solution preparation step, a second step of the primary growth solution preparation step, a third step of the primary growth solution preparation step, a fourth step of the primary growth solution preparation step, a fifth step of the primary growth solution preparation step, and a sixth step of the primary growth solution preparation step, in which: in the first step of the primary growth solution preparation step, the CTAB solution is provided; in the second step of the primary growth solution preparation step, the potassium bromide solution is added to the CTAB solution; in the third step of the primary growth solution preparation step, a silver(I) nitrate solution is added to the mixed solution obtained in the second step of the primary growth solution preparation step; in the fourth step of the primary growth solution preparation step, the gold(III) chloride solution is added to the mixed solution obtained in the third step of the primary growth solution preparation step; in the fifth step of the primary growth solution preparation step, an L-ascorbic acid solution is added to the mixed solution obtained in the fourth step of the primary growth solution preparation step; and in the sixth step of the primary growth solution preparation step, the mixed solution obtained in the fifth step of the primary growth solution preparation step is added to the mixed solution (seed solution) obtained in the fifth seed solution preparation step and then stirred.

In the primary growth solution preparation step, the concentration ratio of CTAB:potassium bromide may be 1:0.05 to 0.15 (e.g., 0.05, 0.06, 0.07, 0.08, 0.09, 0.10, 0.11, 0.12, 0.13, 0.14, or 0.15, or within a range between any two of the values illustrated herein. In the primary growth solution preparation step, the concentration ratio of CTAB:silver(I) nitrate may be 1:0.001 to 0.003 (e.g., 0.0015, 0.0016, 0.0017, 0.0018, 0.0019, 0.0020, 0.0021, 0.0022, 0.0023, 0.0024, 0.0025, 0.0026, 0.0027, 0.0028, 0.0029, or 0.0030, or within a range between any two of the values illustrated herein). In the primary growth solution preparation step, the concentration ratio of CTAB gold(III) chloride is 1:0.005 to 0.015 (e.g., 0.005, 0.006, 0.007, 0.008, 0.009, 0.010, 0.011, 0.012, 0.013, 0.014, or 0.015, or within a range between any two of the values illustrated herein). In the primary growth solution preparation step, the concentration ratio of CTAB:L-ascorbic acid may be 1:0.005 to 0.015 (e.g., 0.005, 0.006, 0.007, 0.008, 0.009, 0.010, 0.011, 0.012, 0.013, 0.014, or 0.015, or within a range between any two of the values illustrated herein). In the primary growth solution preparation step, the concentration ratio of CTAB:gold in the seed solution may be 1:0.5×10⁻⁶ to 5.5×10⁻⁶ (e.g., 0.5×10⁻⁶, 1.5×10⁻⁶, 2.0×10⁻⁶, 2.5×10⁻⁶, 3.0×10⁻⁶, 3.5×10⁻⁶, 4.0×10⁻⁶, 4.5×10⁻⁶ or 5.0×10⁻⁶, or 5.5×10⁻⁶ or within a range between any two of the values illustrated herein).

### Secondary Growth Solution Preparation Step

The secondary growth solution preparation step includes a first step of the secondary growth solution preparation step, a second step of the secondary growth solution preparation step, and a third step of the secondary growth solution preparation step, in which: in the first step of the secondary growth solution preparation step, the L-ascorbic acid solution is added to the mixed solution (primary growth solution) obtained in the sixth step of the primary growth solution preparation at an inflow rate of 0.5 to 2.0 mL/h (e.g., 0.50, 0.75, 1.00, 1.25, 1.50, 1.75, 2.00, 2.25, 2.50, 2.75, or 3.00 mL/h, or within a range between any two of the values illustrated herein) while stirring the primary growth solution; in the second step of the secondary growth solution preparation step, the mixed solution obtained in the first step of the secondary growth solution preparation step is stirred for 1, 2, 4, 6, 8, 10, 12, 14, 16, 18, or 20 minutes, or within a range between any two of the values illustrated herein; and in the third step of the secondary growth solution preparation step, the mixed solution obtained in the second step of the secondary growth solution preparation step leaves to stand for 12, 18, 24, 30, or 36 hours or a range between any two of the values illustrated herein.

In the secondary growth solution preparation step, the concentration ratio of gold:ascorbic acid in the primary growth solution may be 1:0.1 to 1.0 (e.g., 0.1, 0.2, 0.3, 0.4, 0.5, 0.6, 0.7, 0.8, 0.9, or 1.0, or within a range between any two of the values illustrated herein).

Coating Step for Covering Gold Nanorod with Silica

For example, when tetraethoxysilane (TEOS) is used as an alkoxysilane, the coating step for covering the gold nanorod with silica includes a first silica-coating step and a second silica-coating step, in which: in the first silica-coating step, a NaOH solution and a MeOH solution in which commercially available gold nanorods or gold nanorods obtained by the method for producing the gold nanorods is dispersed are mixed while stirring; and in the second silica-coating step, the mixed solution obtained in the first silica-coating step and the MeOH solution in which TEOS is dissolved are mixed while stirring. The concentration ratio of the gold nanorods:NaOH in the first silica-coating step may be 1:0.8 to 1.5 (e.g., 0.8, 1.0, 1.1, 1.2, 1.3, 1.4, or 1.5 or a range between any two of the values illustrated herein). The concentration ratio of the gold nanorods:TEOS in the second silica-coating step may be 1:3 to 15 (e.g., 3, 4, 5, 6, 7, 8, 9, 10, 11, 12, 13, 14, or 15, or a range between any two of the values illustrated herein). The coating step for covering gold nanorod with silica may have a heating and standing step, in which in the heating and standing step, the mixed solution allows to stand for 12, 18, 24, 30, or 36 hours or within a range between any two of the values illustrated herein after the second silica-coating step under temperature condition of 20° C. to 30° C.

In the second silica-coating step, the MeOH solution in which TEOS is dissolved may be added to the MeOH solution in which the gold nanorods are dispersed in two to four installments every 20 to 40 minutes. In the second silica-coating step, the MeOH solution in which the gold nanorods are dispersed may be 20, 22, 24, 26, 28, or 30% MeOH solution or may be MeOH solution in a range between any two of the values illustrated herein. The stirring time in the second silica-coating step may be 10, 15, 20, 25, 30, 35, or 40 minutes or within a range between any two of the values illustrated herein.

A method disclosed in JP2018-127699A may be used to the method for producing the gold nanorods and the method for coating the gold nanorod with silica.

### EXAMPLES

### Example 1

### Synthesis of Au Nanorods (AuNRs)

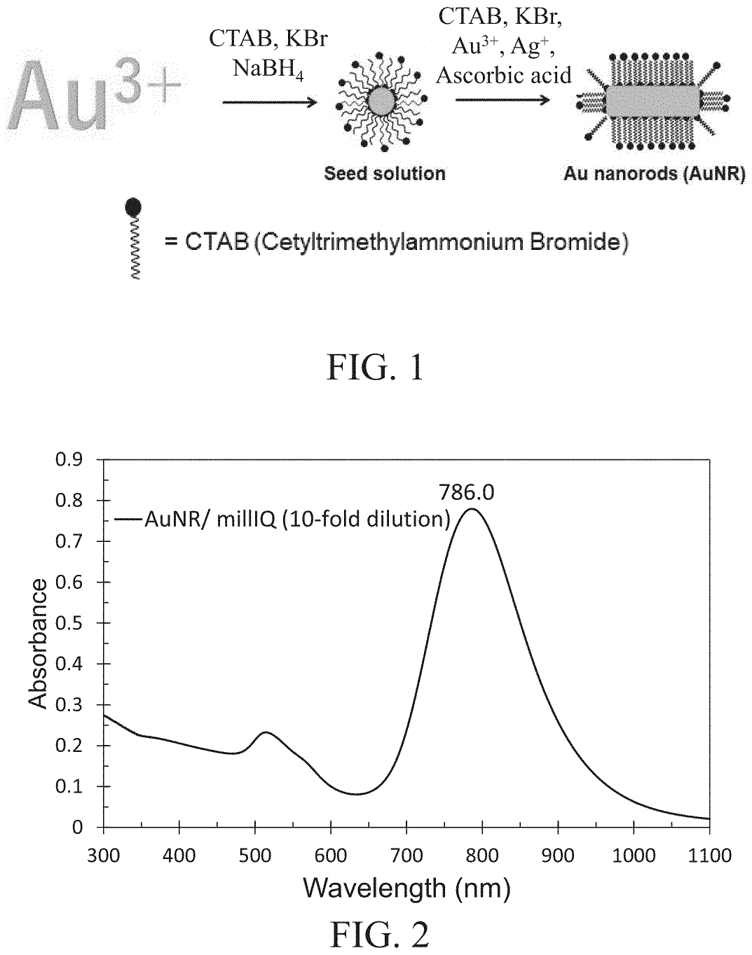

A seed-mediated method was used to synthesize hexadecyltrimethylammonium bromide (CTAB) protected AuNRs. The synthesis scheme is shown in FIG. 1.

Samples Used are as Follows
milliQ (used at 30° C. in a thermostatic chamber)
Hexadecyltrimethylammonium bromide (CTAB) (Nacalai Tesque, MW=364.45)

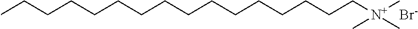 [SMILES: CCCCCCCCCCCCCCCC[N+](C)(C)C ; [Br-]]

Potassium bromide (KBr) (Kishida Chemical, MW=119.0)
Gold(III) chloride tetrahydrate (HAuCl₄) (Nacalai Tesque, MW=411.85)
Sodium hydrogenide (NaBH₄) (Nacalai Tesque, MW=37.83)
Silver(I) nitrate (AgNO₃) (Wako Chemicals, MW=169.87)
L-Ascorbic acid (AA) (Nacalai Tesque, MW=176.13)

The Samples were Adjusted as Follows

### <Seed Solution>

0.125M CTAB aq.

It was prepared by dissolving CTAB (0.3645 g, 1.0 mmol) in milliQ (8.0 mL).
0.1M KBr aq.

It was prepared by dissolving KBr (120.5 mg, 1.0 mmol) in milliQ (10 mL).
2.4 mM HAuCl₄ aq.

It was prepared by diluting 4.6 mM HAuCl₄ aq.
10 mM NaBH₄ aq.

It was prepared by dissolving NaBH₄ (3.8 mg, 0.1 mmol) in ice-cold milliQ (10 mL).

### <Primary Growth Solution>

0.122M CTAB aq.

It was prepared by dissolving CTAB (3.4314 g, 9.4 mmol) in milliQ (77 mL).
0.9412 M KBr aq.

It was prepared by dissolving KBr (1.1203 g, 9.4 mmol) in milliQ (10 mL).
19.2 mM AgNO₃ aq.

It was prepared by dissolving AgNO₃ (39.3 mg, 0.23 mmol) in milliQ (12.05 mL).
4.6 mM HAuCl₄ aq.

It was prepared by dissolving HAuCl₄.4H₂O (255.49 mg, 0.62 mmol) in milliQ (134.858 mL).
0.105 M AA aq.

It was prepared by dissolving AA (186.5 mg, 1.1 mmol) in milliQ (10 mL).

### <Secondary Growth Solution>

9.48 mM AA aq.

It was prepared by dissolving (16.7 mg, 94.8 mol) in milliQ (10 mL).

### 1-1 Preparation of Seed Solution

0.125 M CTAB aq. (8.0 mL, 1.0 mmol), 0.1 M KBr aq. (1.0 mL, 0.1 mmol), and 2.4 mM HAuCl₄.4H₂O aq. (1.0 mL, 2.4 mol) were added into a 14 mL glass sample bottle in this order. Then, the mixture was stirred vigorously at room temperature. 10 mM NaBH₄ aq. (0.6 mL, 6.0 mol) was added to the bottle and the stirring was continued for 2 min. The stirring was stopped, and the mixture was allowed to stand still for 3 min. After seven times of inversion mixing, it was allowed to stand for 1 h in a water bath at 30° C. The total volume of the seed solution was 10.6 mL and the final concentration thereof was as follows.

### Final Concentration

[CTAB]=94.34 mM
[KBr]=9.43 mM
[HAuCl₄.4H₂O]=0.226 mM
[NaBH₄]=0.556 mM

### 1-2 Preparation of Primary Growth Solution

0.122 M CTAB aq. (77 mL, 9.39 mmol) was added into a 250 mL medium bottle and stirred at room temperature. 0.9412 M KBr aq. (1.0 mL, 0.9412 mmol) and 19.2 mM AgNO₃ aq. (1.0 mL, 19.2 mol) were added into the bottle in this order, and then 4.6 mM HAuCl₄.4H₂O aq. (20 mL, 92 mol) and 0.105 M AA aq. (1.0 mL, 0.105 mmol) were added into the bottle in this order. 0.135 mL of the seed solution (Au seeds), which had been allowed to stand for exactly one hour, was added to this solution, and the mixture was stirred vigorously. The total volume of the primary growth solution is 100.135 mL and the final concentration is as follows.

### Final Concentration

[CTAB]=94.0 mM
[KBr]=9.40 mM
[AgNO₃]=0.192 mM
[HAuCl₄.4H₂O]=0.919 mM
[AA]=1.05 mM
[Au seed]=0.305 μM

### 1-3 Preparation of Secondary Growth Solution

9.48 mM AA aq. (5.00 mL, 47.4 μmol) was added to the first growth solution with vigorous stirring at room temperature. The AA aq was added to the solution by using a microsyringe pump (AS ONE MSP-1D, syringe inner diameter: 17.0 mm, inflow volume: 5.00 mL, inflow rate: 1.75 mL/h, Termo syringe 10 mL ss-10Sz (plastic)). The stirring was continued for 10 min and then the stirring was stopped. The total volume (105.135 mL) was transferred to a 200 mL medium bottle and kept in an incubator at 25° C. for 24 hours. The total volume of the secondary growth solution was 100.135 mL and the final concentration was as follows.
[AA]=0.451 mM
[Au]=0.88 mM

### 1-4 AuNR Purification

After being allowed to stand for 24 hours, the solution including AuNRs (105.135 mL) were divided into 6 parts (about 17.5 mL), each of which was dispensed into six 50 mL plastic centrifuge tubes. Subsequently, centrifugation (10,000 rpm [9,840×g], 30 min, 25° C.) was performed. The supernatant of each solutions was removed, and the precipitates were redistributed equally in milliQ. The prepared solution was designated as AuNR/milliQ.

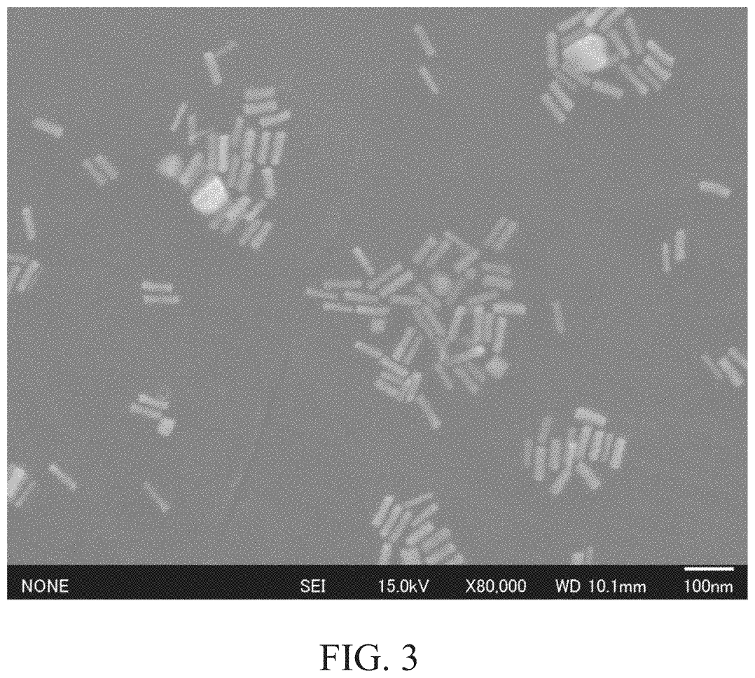

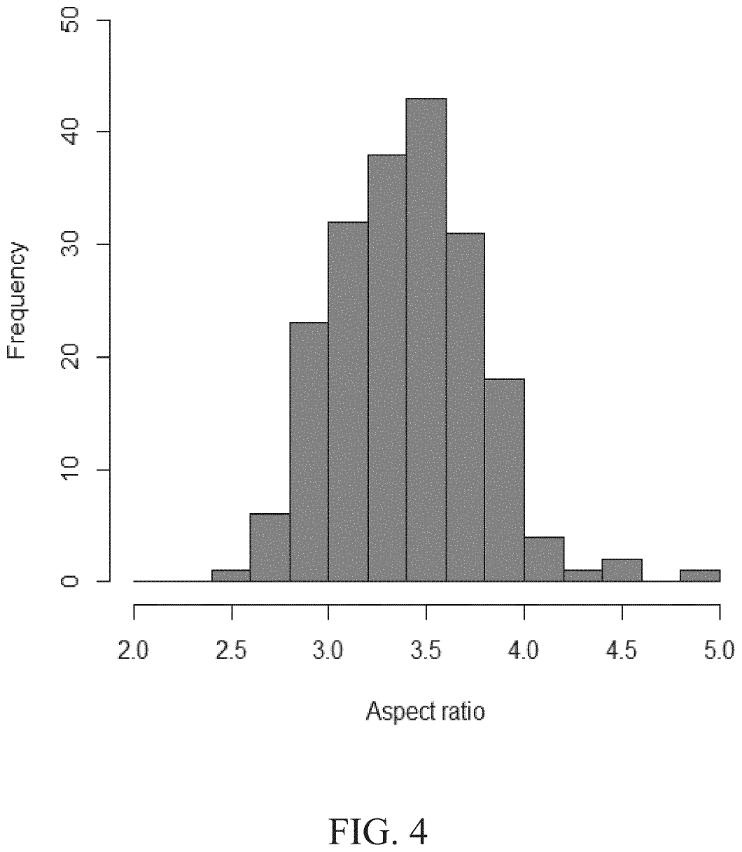

Absorption spectrum of the prepared AuNR (PMMA cell, optical path length: 1 cm, [AuNR]=0.0736 nM) was measured. The result is shown in FIG. 2. As shown in FIG. 2, the peak of the maximum absorption wavelength was observed at 786 nm. The AuNRs were observed using a field emission scanning electron microscopy (FE-SEM) (FIG. 3), and the particle size distribution of the AuNRs (n=200) was further calculated from the FE-SEM photograph (FIG. 4). The distribution result is shown in Table 1.

TABLE 1

| Long axis | 66.7 ± 4.54 nm |
|---|---|
| Short axis | 19.7 ± 1.95 nm |
| Aspect ratio | 3.40 ± 0.370 |

### 1-5 Discussion

From these results, the AuNRs were successfully synthesized by the FE-SEM observation. In addition, the absorption spectrum of the AuNRs was observed in the near-infrared region.

### Example 2

### Silica Coating of AuNR (AuNR@TEOS)

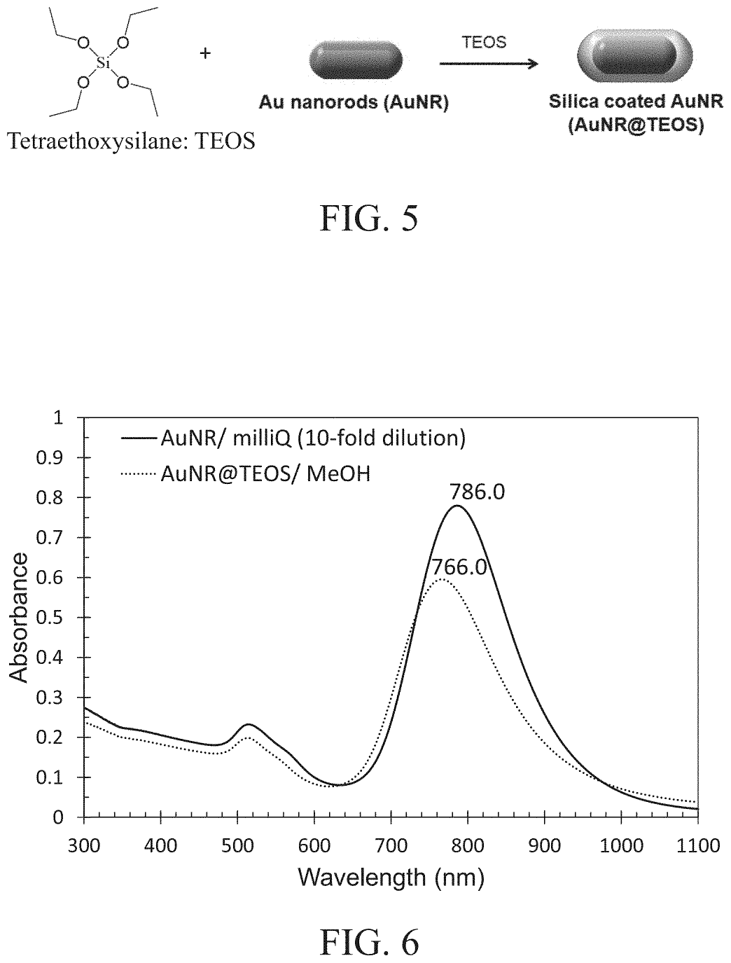

Silica-coated AuNR (AuNR@TEOS) was prepared using tetraethoxysilane (TEOS). The preparation scheme is shown in FIG. 5.

The sample used are as follows.
AuNR/milliQ ([Au]=0.88 mM, [AuNR]=0.736 nM)
Tetraethoxysilane (TEOS) (MW=208.33) (0.934 g/mL)
milliQ
MeOH
Sodium hydroxide (NaOH) (MW=40.0)

The sample was adjusted as follows
0.1 M NaOH aq.

It was prepared by dissolving NaOH (94.7 mg, 2.4 mmol) in milliQ (23.7 mL).
20 vol % (0.89 M) TEOS/MeOH

It was prepared by mixing TEOS (100 μL, 0.45 mmol) and MeOH (400).

### 2-1 Preparation of AuNR@TEOS

The AuNR/milliQ (5.0 mL) was added into a 15 mL PP centrifuge tube and centrifuged (8,000 rpm [6,011×g], 30 min, 25° C.). After the centrifugation, 1.25 mL of the supernatant was removed, and the precipitate was redistributed by addition of MeOH (1.25 mL). The prepared solution was designated as AuNR/25% MeOH aq. The total volume of the solution is 5.0 mL and the final concentration is [Au]=0.88 mM.

The total volume of the AuNR/25% MeOH aq. (50 mL) was added into a 30 mL PP wide-mouthed bottle (film case) and stirred at room temperature. 0.1 M NaOH aq. (50 μL, 5.0 mol) was added to the AuNR/25% MeOH aq. 20 vol % TEOS/MeOH (15 μL, 13.4 μmol) was added to the mixed solution 3 times every 30 minutes, stirred for 30 minutes, and then allowed to stand in an incubator at 25° C. for 24 hours. The total volume of the solution is 5.095 mL and the final concentration is as follows.

### Final Concentration

[Au]=0.864 mM
[NaOH]=0.98 mM
[TEOS]=7.86 mM

After being allowed to stand for 24 hours, the total volume of the prepared solution (5.095 mL) was added into a 15 mL PP centrifuge tube and centrifuged (8,000 rpm [6,011×g], 30 min, 25° C.). The supernatant was removed, and the precipitate was redistributed equally with MeOH. Again, it was centrifuged (6,000 rpm [3,381×g], 30 min, 25° C.), the supernatant was removed, and the precipitate was filled up to 5.0 mL with MeOH. After that, the total volume of the solution (5.0 mL) was added into a 14 mL glass sample bottle, and the prepared solution was designated as AuNR@TEOS/MeOH. The total volume of the solution is 5.0 mL and the final concentration is [Au]=0.88 mM.

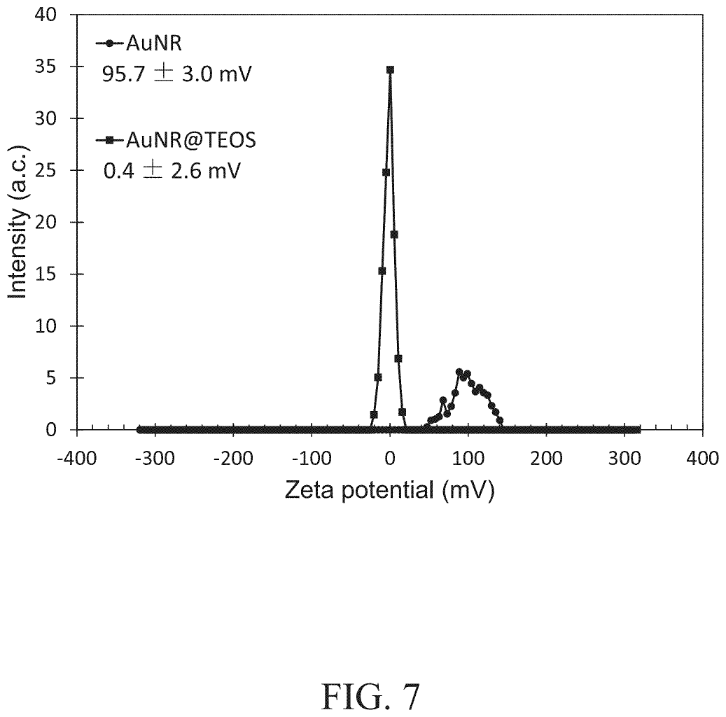

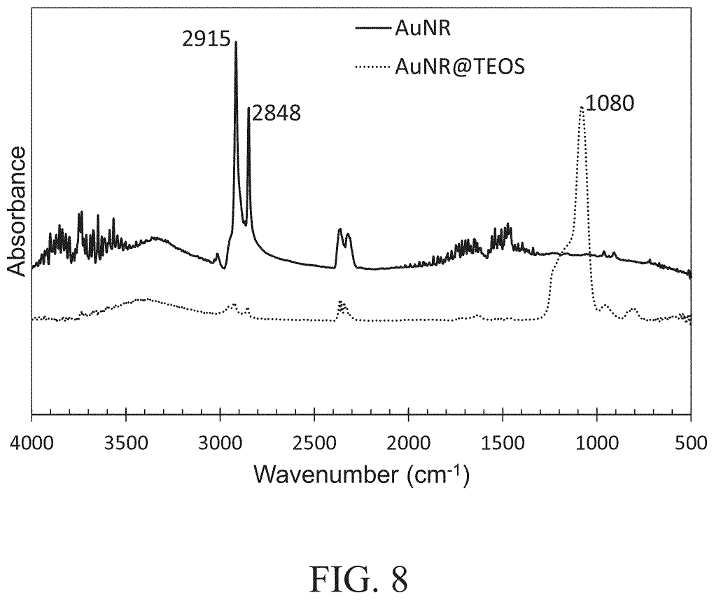

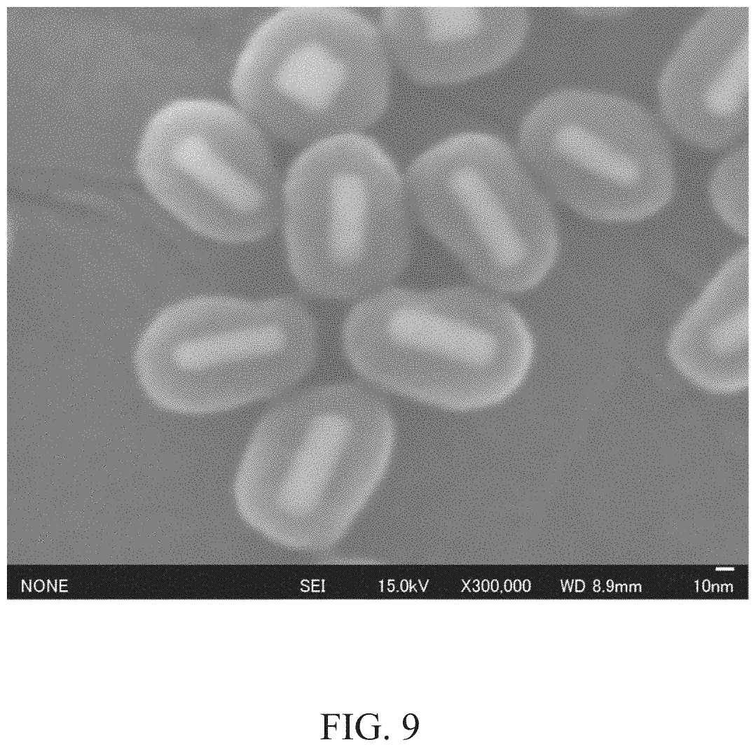

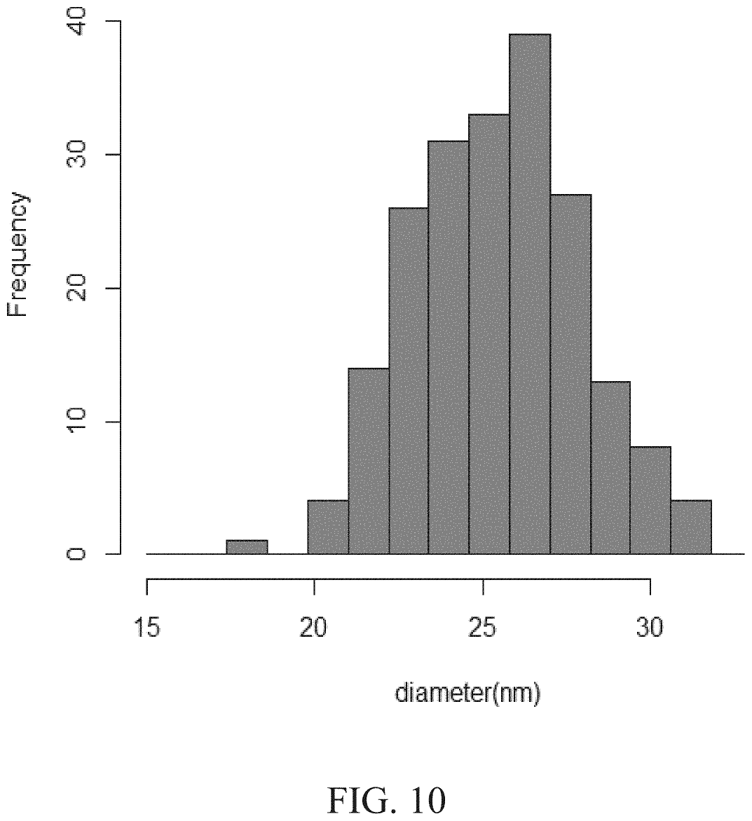

Absorption spectra, Zeta potentials and spectra of Fourier transform infrared spectroscopy (FT-IR) of AuNR and AuNR@TEOS were measured (FIGS. 6, 7 and 8, respectively). In addition, AuNRs@TEOS was observed using FE-SEM (FIG. 9), and furthermore, the silica layer distribution of AuNRs@TEOS (n=200) was calculated from the FE-SEM photograph (FIG. 10). The distribution result is shown in Table 2.

TABLE 2

| | |
|---|---|
| Long axis | 63.2 ± 4.56 nm |
| Short axis | 19.7 ± 1.94 nm |
| Aspect ratio | 3.23 ± 0.335 |
| Silica layer | 25.4 ± 2.46 nm |

FIG. 10 shows that the silica layer of AuNR@TEOS has a thickness of at least 15 nm.

### 2-2 Discussion

FE-SEM observations showed that silica was coated on the AuNR surface. Therefore, the absorption spectra measurements showed a shift in the maximum absorption wavelength due to a change in the local refractive index of the particle surface. In addition, Zeta potential measurements indicated that silica-derived negatively charged hydroxy groups are introduced into the particle surface by coating the positively charged CTAB-protected AuNR surface with silica, resulting in a shift of the Zeta potential to a negative value. FT-IR measurements showed a new Si—O bond-derived peak around 1100 cm⁻¹ which did not appear in the CTAB-protected AuNR. These results indicate that the AuNRs@TEOS (silica coating) were produced.

### Comparative Experimental Example A

### Introduction of 3-aminopropyltriethoxysilane into AuNR

AuNR@APTES was prepared by introducing 3-aminopropyltriethoxysilane (APTES) into AuNR.

Samples Used are as Follows.
AuNR/milliQ ([Au]=0.88 mM, [AuNR]=0.736 nM)
3-Aminopropyltriethoxysilane (APTES) (MW=221.37) (0.946 g/mL)
milliQ
MeOH
Sodium hydroxide (NaOH) (MW=40.0)

The samples were adjusted as follows.
0.1 M NaOH aq.

It was prepared by dissolving NaOH (94.7 mg, 2.4 mmol) in milliQ (23.7 mL).
10 vol % (0.427 M) APTES/MeOH

It was prepared by mixing APTES (50 μL, 0.21 mmol) and MeOH (450 μL).

### A-1 Preparation of AuNR@ APTES

AuNR/milliQ (5.0 mL) was added into a 15 mL PP centrifuge tube and centrifuged (8,000 rpm [6,011×g], 30 min, 25° C.). After the centrifugation, 1.25 mL of the supernatant was removed. The solution was redistributed with MeOH (1.25 mL). The prepared solution was designated as AuNR/25% MeOH aq. The total volume of the solution is 5.0 mL and the final concentration is [Au]=0.88 mM.

The total volume of AuNR/25% MeOH aq. (5.0 mL) was added into a 30 mL PP wide-mouthed bottle (film case) and stirred at room temperature. 0.1 M NaOH aq. (50 μL, 5.0 mol) was added to AuNR/25% MeOH aq. 10 vol % (0.427 M) APTES/MeOH (15 μL, 6.40 mol) was added to the mixed solution twice every 30 minutes, stirred for 30 minutes, and then allowed to stand in an incubator at 25° C. for 24 hours. The total volume of the solution was 5.1 mL and the final concentration was as follows.

### Final Concentration

[Au]=0.863 mM
[NaOH]=0.98 mM
[APTES]=4.19 mM

After 24 hours of standing, the total volume of the prepared solution (5.1 mL) was added into a 15 mL PP centrifuge tube and centrifuged (8,000 rpm [6,011×g], 30 min, 25° C.). The supernatant was removed, and the precipitate was redistributed equally with MeOH. Again, it was centrifuged (6,000 rpm [3,381×g], 30 min, 25° C.), the supernatant was removed, and the precipitate was filled up to 5.0 mL with MeOH. Thereafter, the total volume of the solution (5.0 mL) was added into a 14 mL glass sample bottle, and the prepared solution was designated AuNR@APTES/MeOH. The total volume of the solution is 5.0 mL and the final concentration is [Au]=0.88 mM.

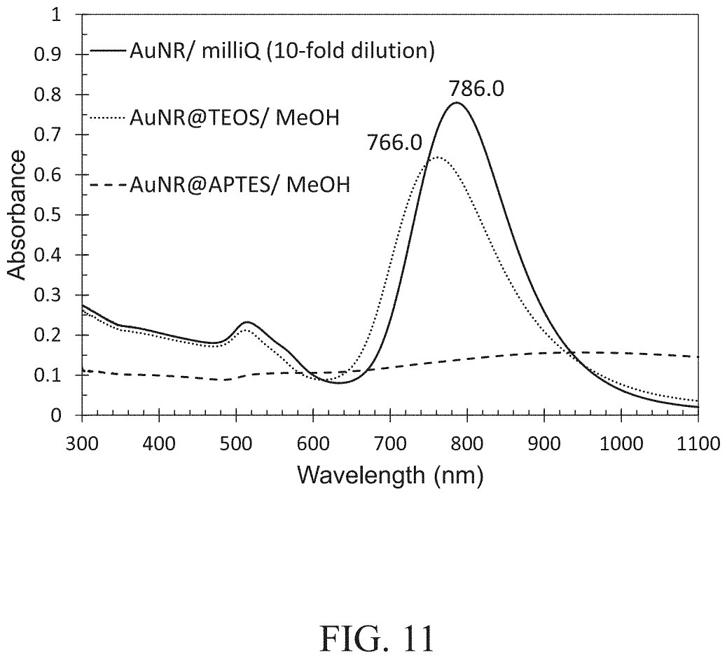

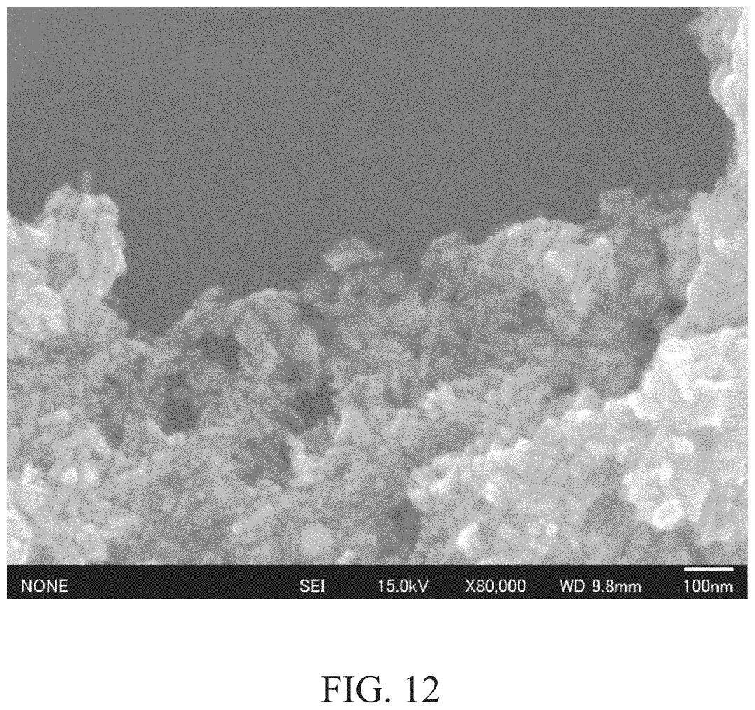

Absorption spectra (glass cell, optical path length 1 mm, [AuNR]=0.736 nM) of AuNR, AuNR@TEOS, and AuNR@APTES were measured (FIG. 11). In addition, AuNR@APTES was observed using FE-SEM (FIG. 12).

### A-2 Discussion

As shown in FIG. 11, the absorption spectrum of AuNR@APTES shows a clear decrease in the absorption band due to the lack of sample dispersion. The FE-SEM photograph in FIG. 12 also revealed that no silica coating was made.

### Example 3

### Synthesis of 3-aminopropyltriethoxysilane Introduced AuNR@TEOS (AuNR@TEOS-APTES)

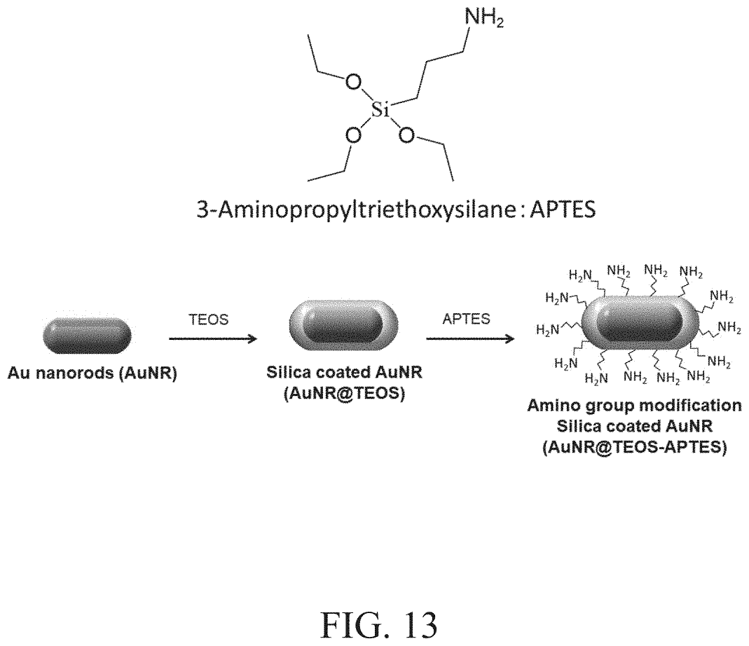

AuNR@TEOS (AuNR@TEOS-APTES) with APTES (—NH₂ group) was prepared using 3-aminopropyltriethoxysilane (APTES). The production scheme is shown in FIG. 13.

Samples Used are as Follows
AuNR@TEOS/MeOH ([Au]=0.88 mM, [AuNR]=0.736 nM)
Tetraethoxysilane (TEOS) (MW=208.33) (0.934 g/mL)
3-Aminopropyltriethoxysilane (APTES) (MW=221.37) (0.946 g/mL)
milliQ
MeOH
Sodium hydroxide (NaOH) (MW=40.0)

The samples were adjusted as follows
0.1 M NaOH aq.

It was prepared by dissolving NaOH (94.7 mg, 2.4 mmol) in milliQ (23.7 mL).
20 vol % (0.89 M) TEOS/MeOH

It was prepared by mixing TEOS (100 μL, 0.45 mmol) and MeOH (400 μL).
10 vol % (0.427 M) APTES/MeOH

It was prepared by mixing APTES (50 μL, 0.21 mmol) and MeOH (450 μL).

### 3-1 Preparation of AuNR@TEOS-APTES

AuNR@TEOS/MeOH (5.0 mL) was added into a 15 mL PP centrifuge tube, centrifuge (8,000 rpm [6,011×g], 30 min, 25° C.). After the centrifugation, 3.75 mL of the supernatant was removed, and the precipitate was redistributed with 3.75 mL of milliQ. The prepared solution was designated AuNR@TEOS/25% MeOH aq. The total volume of the solution is 5.0 mL and the final concentration is [Au]=0.88 mM.

The total volume of the AuNR@TEOS/25% MeOH aq. (50 mL) was added into a 30 mL PP wide-mouthed bottle (film case) and stirred at room temperature. 0.1 M NaOH aq. (50 μL, 5.0 mol) was added to the AuNR@TEOS/25% MeOH aq. 10 vol % (0.427 M) of APTES/MeOH (50 μL, 21.4 mol) was added to the mixture, and the stirring was continued for 5 minutes. Next, the mixture was stirred for 5 hours in a water bath at 50° C. The total volume of the solution was 5.1 mL and the final concentration was as follows.

### Final Concentration

[Au]=0.863 mM
[NaOH]=0.98 mM
[APTES]=4.19 mM

After 5 hours of agitation, the total volume of the prepared solution (5.1 mL) was added into a 15 mL PP centrifuge tube, centrifuged (8,000 rpm [6,011×g], 30 min, 25° C.). After the centrifugation, the supernatant was removed, and the precipitate was redistributed equally with MeOH. Again, centrifugation (6,000 rpm [3,381×g], 30 min, 25° C.) was carried out and the supernatant was removed, and the precipitate was filled up to 5.0 mL with MeOH. The total volume of the prepared solution (5.0 mL) was then added into a 10 mL glass sample bottle, and the prepared solution was designated as AuNR@TEOS-APTES/MeOH. The total volume of the solution was 5.0 mL and the final concentration was as follows.

### Final Concentration

[Au]=0.88 mM (theoretical value)
[—NH₂]=4.27 mM (theoretical value)

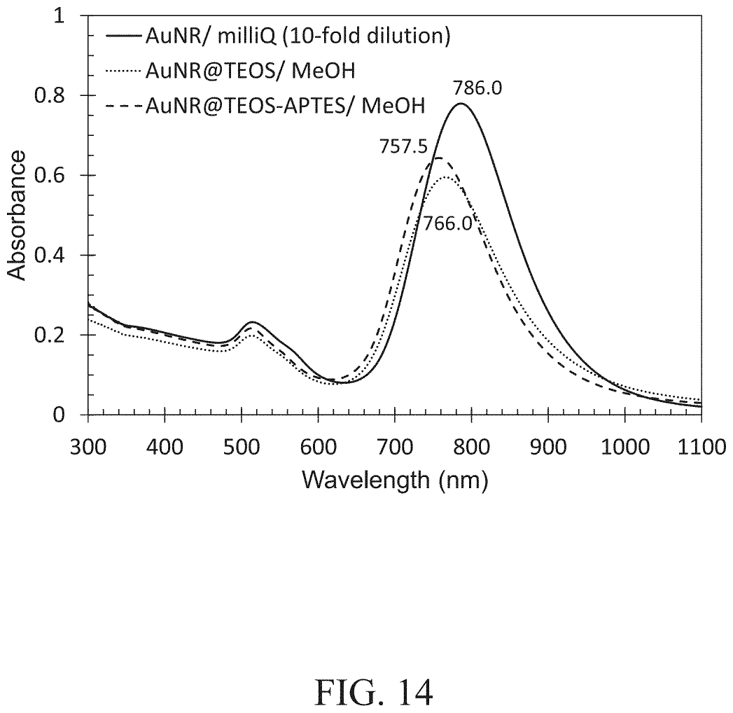

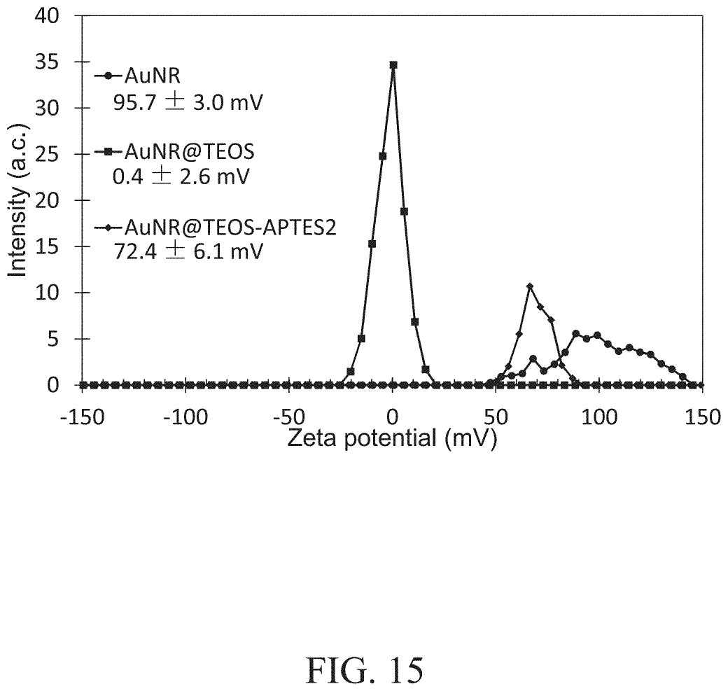

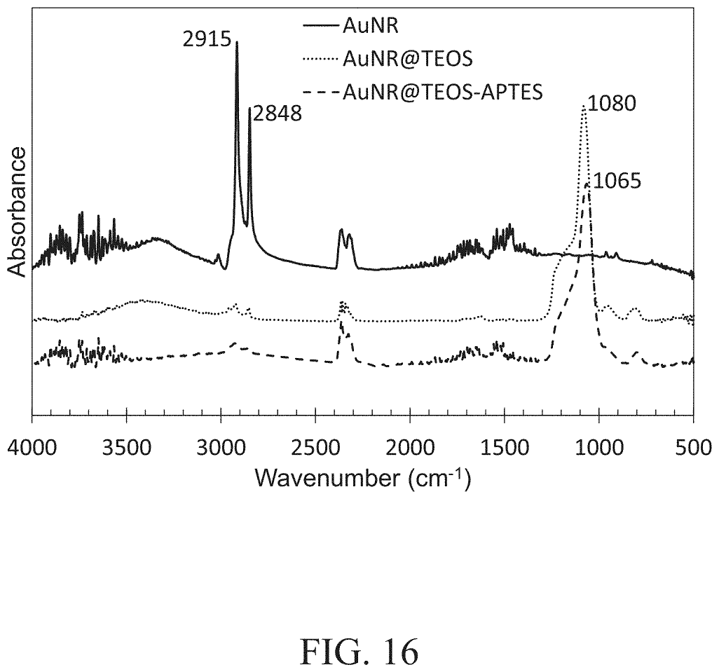

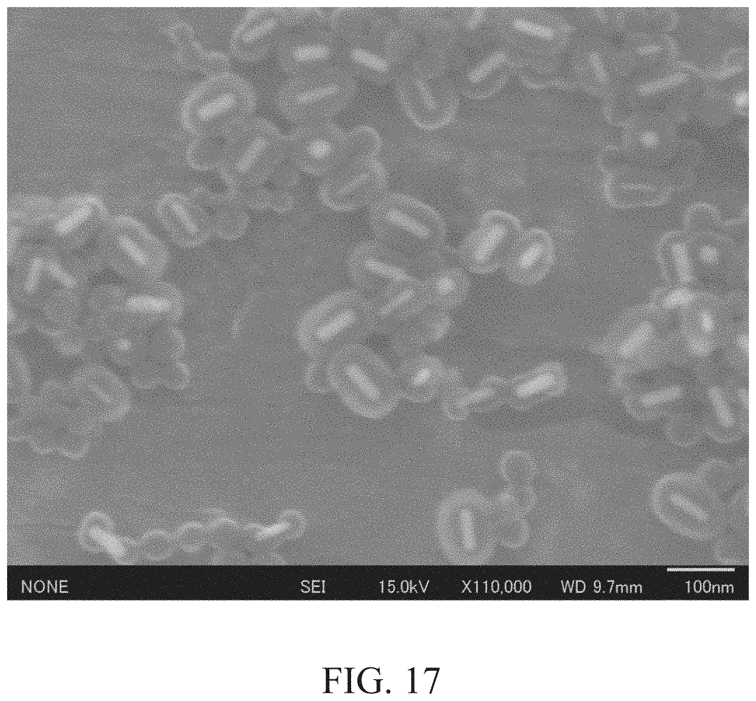

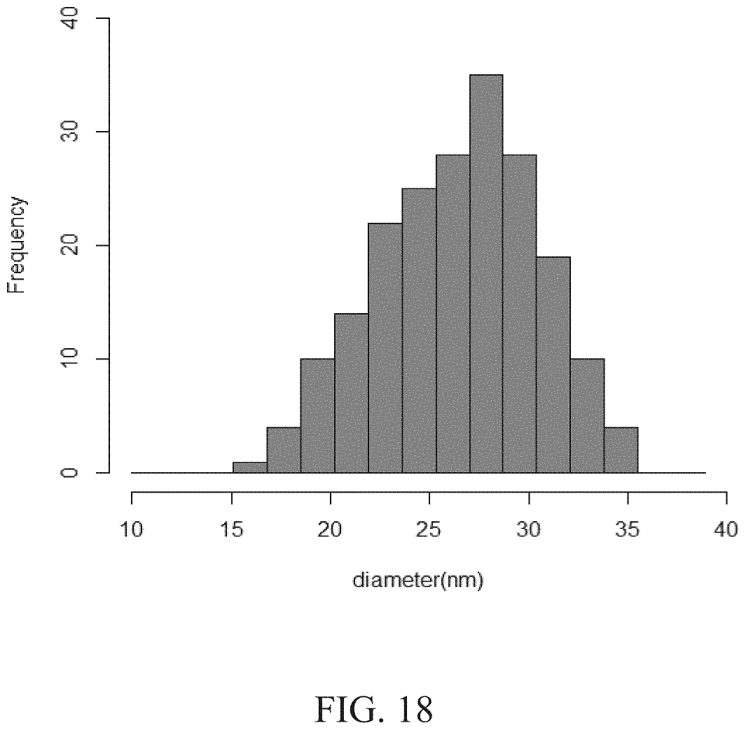

Absorption spectra, Zeta potential and spectra of FT-IR of AuNR, AuNR@TEOS and AuNR@TEOS-APTES were measured (FIG. 14, FIG. 15 and FIG. 16, respectively). In addition, AuNR@TEOS-APTES was observed using FE-SEM (FIG. 17), and the silica layer distribution of AuNR@TEOS-APTES (n=200) was calculated from the FE-SEM photograph (FIG. 18). The distribution results are shown in Table 3.

TABLE 3

| | |
|---|---|
| Long axis | 64.9 ± 5.05 nm |
| Short axis | 21.0 ± 2.27 nm |
| Aspect ratio | 3.13 ± 0.381 |
| Silica layer | 26.4 ± 4.01 nm |

FIG. 18 shows that the silica layer of AuNR@TEOS-APTES is at least 15 nm thick.

### 3-2 Discussion

As a result of the absorption spectrum measurement, the maximum absorption wavelength was shifted. It is considered that this was caused by the change in the local refractive index due to the particle surface newly modified with amino group. Furthermore, the Zeta potential showed that while the particle surface was negatively charged due to the hydroxy groups from the silica coating, the charge of the surface newly modified with the amino group is positively shifted. These results suggest that the amino groups were introduced. The FE-SEM observations showed no change in the particle size.

### Example 4

### Modification of Dansyl Group to AuNR@TEOS-APTES

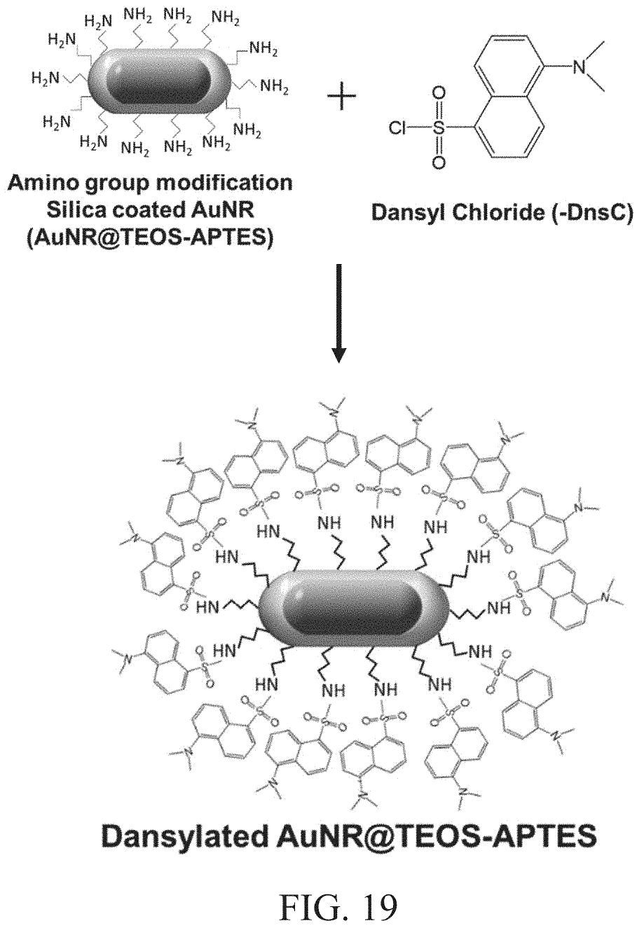

AuNR@TEOS-APTES modified with the Dansyl group (AuNR@TEOS-APTES-Dansyl) was produced using Dansyl chloride. The production scheme is shown in FIG. 19.

Samples Used are as Follows
AuNR@TEOS-APTES/MeOH ([Au]=0.88 mM, [AuNR]=0.736 nM, [—NH₂]=4.27 mM (theoretical value))
Dansyl Chloride (MW=269.75)
CH₂Cl₂
MeOH
Triethylamine

The samples were adjusted as follows
Dry CH₂Cl₂

An appropriate amount of CH₂Cl₂ (100 mL) and CaCl₂) were added into a 200 mL eggplant flask and the flask was covered with a glass stopper. The mixture was then shaken well and allowed to stand overnight at room temperature. After being allowed to stand, dry CH₂Cl₂ was obtained by distillation in a nitrogen atmosphere. The obtained dry CH₂Cl₂ was added into an eggplant flask and the flask was covered with septum to prevent it from being exposed to air.
2.38 mM Dansyl chloride/dry CH₂Cl₂

It was prepared by dissolving Dansyl chloride (5.76 mg, 21.4 mol) in dry CH₂Cl₂ (9.0 mL).

AuNR@TEOS-APTES/MeOH (1.0 mL) was added into a 1.5 mL eppendorf tube, and centrifuged (8,000 rpm [5,796×g], 30 min, 25° C.). The supernatant was removed, and the solution was redistributed equally with dry CH₂Cl₂. Triethylamine (3.0 μL, 21.4 mol) was added to the prepared AuNR@TEOS-ATPES/dry CH₂Cl₂, and the total volume (approximately 1.0 mL) was added into a 100 mL two-mouth eggplant flask and stirred under nitrogen atmosphere. Heat reflux was started after addition of the total volume of 2.38 mM Dansyl chloride/dry CH₂Cl₂ (9.0 mL). After 8 hours, the stirring and heat reflux were stopped. The total volume of the solution was 10 mL and the final concentration was as follows.

### Final Concentration

[Au]=0.088 mM
[—NH₂]=0.427 mM
[Dansyl chloride]=2.14 mM
[triethylamine]=2.14 mM

Two 15 mL PP centrifuge tubes containing 5.0 mL of the prepared solution in each centrifuge tube were prepared. These centrifuge tubes were centrifuged (8,000 rpm [6,011×g], 30 min, 25° C.), the supernatants were removed, and the precipitates were redistributed equally with CH₂Cl₂. This centrifugation step was repeated 3 times. After that, these centrifuge tubes were centrifuged (8,000 rpm [6,011×g], 30 min, 25° C.), the supernatants were removed, and the precipitates were redistributed equally with MeOH. This centrifugation step was repeated 3 times. The prepared solution was designated as AuNR@TEOS-APTES-Dansyl/MeOH. The total volume of the solution is 10 mL and the final concentration is as follows.

### Final Concentration

[Au]=0.088 mM
[—NH-Dansyl]=0.427 mM

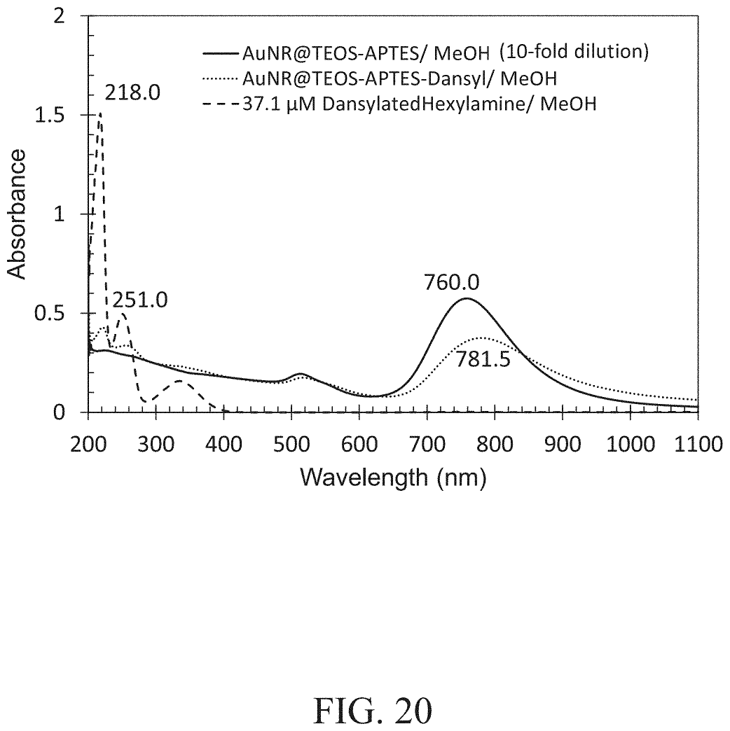

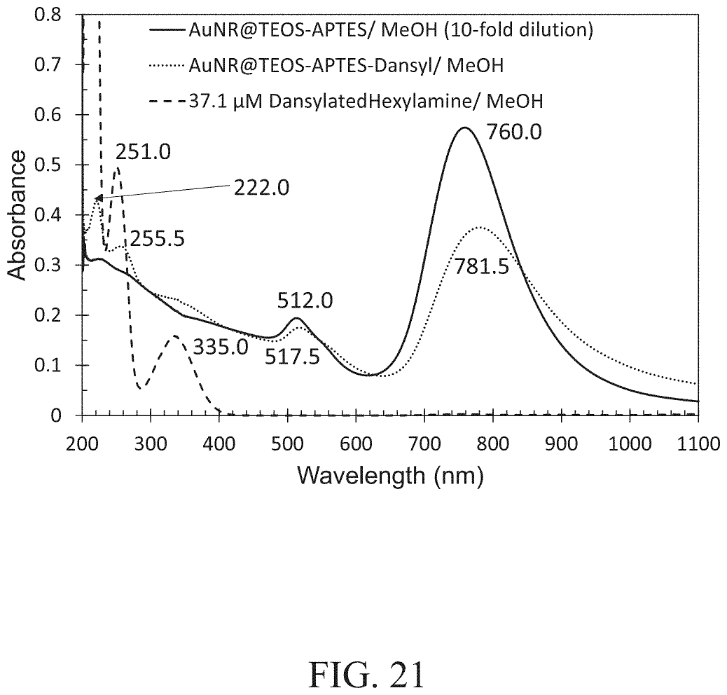

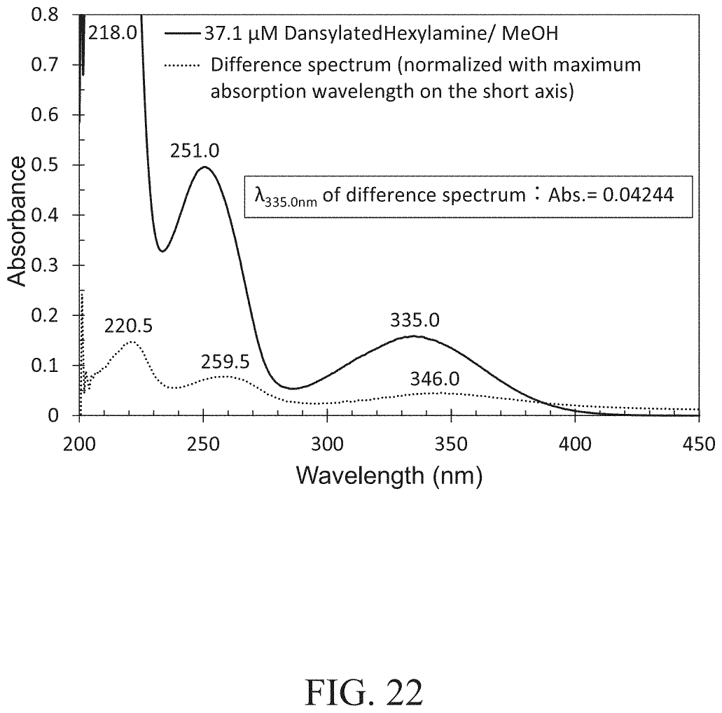

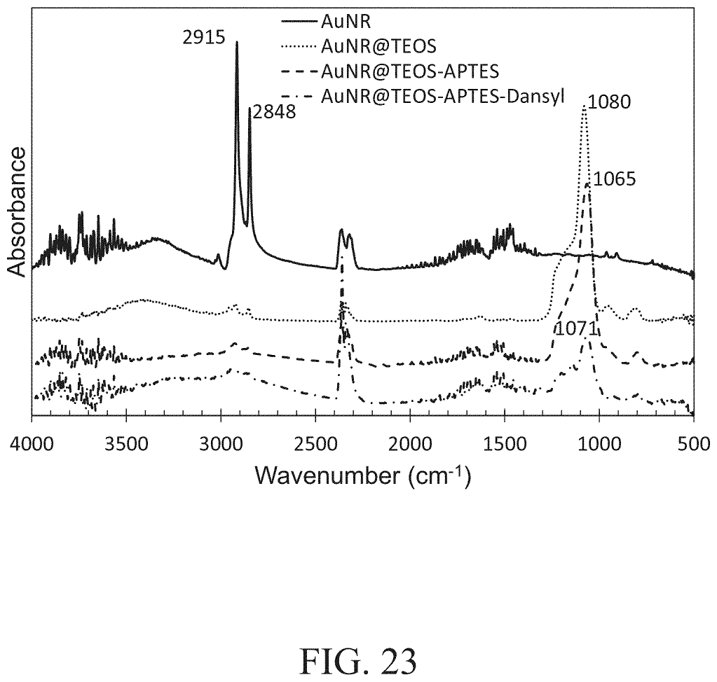

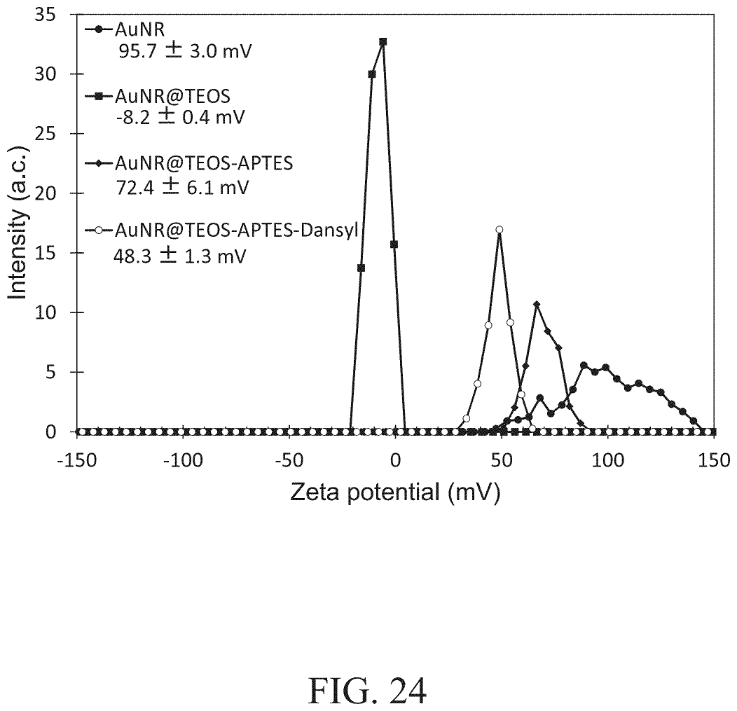

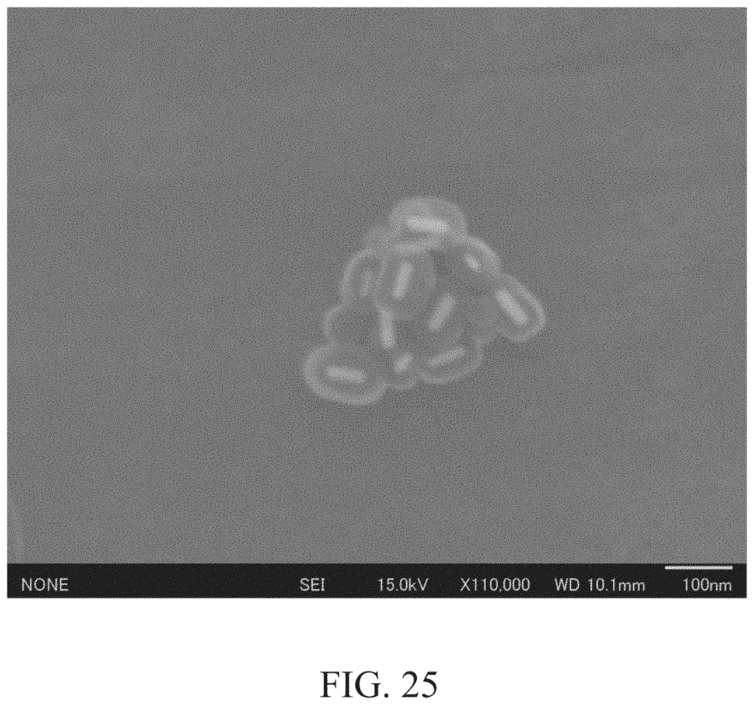

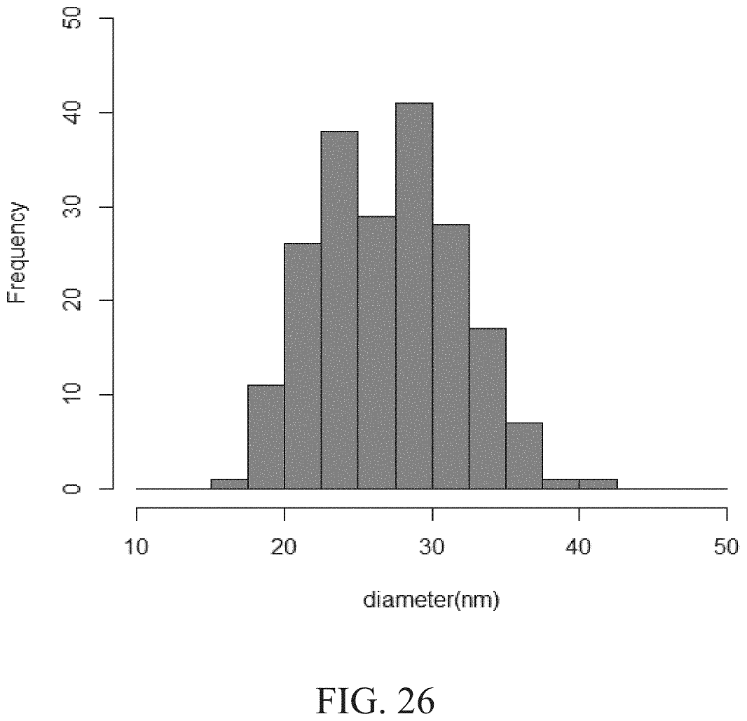

Absorption spectra of AuNR@TEOS-APTES, AuNR@TEOS-APTES-Dansyl and Dansylated hexylamine (FIGS. 20 and 21), a spectrum representing the difference in absorption spectra between AuNR@TEOS-APTES and AuNR@TEOS-APTES-Dansyl (that is, the difference before and after Dansyl group modification) (FIG. 22), and spectra of FT-IR and Zeta potentials of AuNR, AuNR@TEOS, AuNR@TEOS-APTES and AuNR@TEOS-APTES-Dansyl (FIGS. 23 and 24, respectively) were measured. In addition, AuNR@TEOS-APTES-Dansyl was observed using the FE-SEM (FIG. 25) and the silica layer distribution (n=200) of AuNR@TEOS-APTES-Dansyl was calculated from the FE-SEM photograph (FIG. 26). The distribution results are shown in Table 4.

TABLE 4

| | |
|---|---|
| Long axis | 64.1 ± 5.81 nm |
| Short axis | 21.1 ± 2.72 nm |
| Aspect ratio | 3.08 ± 0.363 |
| Silica layer | 27.0 ± 4.67 nm |

FIG. 18 shows that the silica layer of AuNR@TEOS-APTES-Dansyl has a thickness of at least 15 nm.

### 4-2 Discussion

The absorption spectra showed new peaks around 220 nm, 250 nm, and 330 nm, which were derived from the Dansyl group. FT-IR measurements showed that the intensity of C—H bond-derived peak was higher than that of Si—O bond-derived peak. This is probably due to the modification of the Dansyl group, which strongly reflects the C—H bond of the Dansyl group in the IR spectra. Zeta potential measurements showed that the positive charge on the surface of the particles due to the binding of the amino group to the Dansyl group was weakened by the binding of the Dansyl group to the amino group, resulting in a shift of the Zeta potential in the negative direction. The modification of the Dansyl group on the surface of the particles was successfully achieved. Furthermore, FE-SEM observation showed no change in the particle size.

### Example 5

### The Number of Dansyl Group Modifications Per a Single Particle of AuNR@TEOS-APTES-Dansyl

The number of Dansyl group modifications per a single particle of the AuNR@TEOS-APTES-Dansyl was calculated with curve fitting using the nonlinear least squares method to estimate the fitting parameters that best fit the data. To reduce the influence of the long axis-derived peaks of AuNR@TEOS-APTES-Dansyl, the curve fitting was performed in the wavelength range of 210-450 nm. The number of Dansyl group modifications per the particle of the AuNR@TEOS-APTES-Dansyl was (3.68±0.77)×104.

### Example 6

### Fluorescence of AuNR@TEOS-APTES-Dansyl

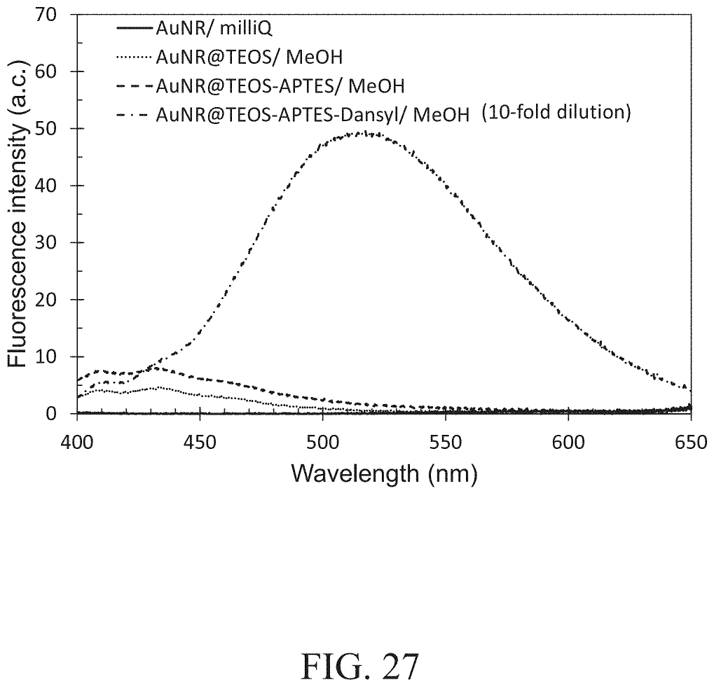

Fluorescence spectral measurements (quartz cell, 1 cm optical path length, Ex: 335 nm) of AuNR, AuNR@TEOS, AuNR@TEOS-APTES and AuNR@TEOS-APTES-Dansyl were measured (FIG. 27). The fluorescence after the Dansyl group modification could be detected by the fluorescence spectroscopic measurements.

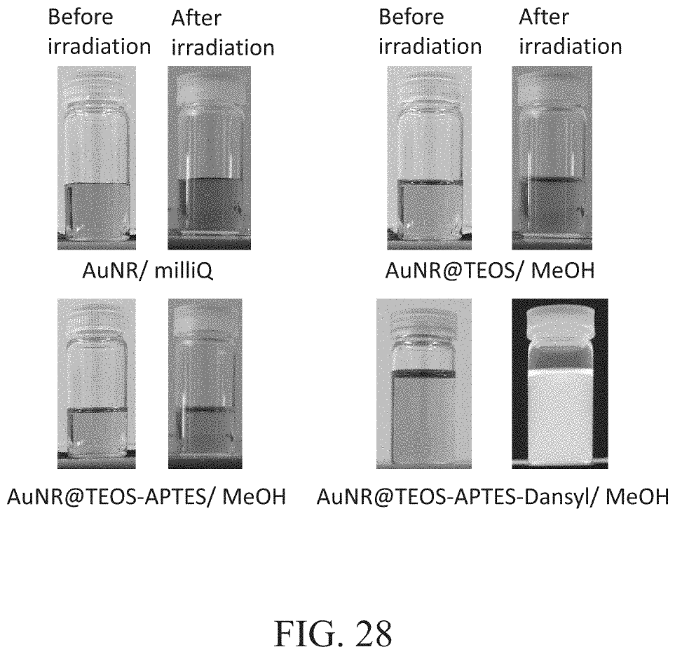

Furthermore, each of AuNR, AuNR@TEOS, AuNR@TEOS-APTES, and AuNR@TEOS-APTES-Dansyl was added into a corresponding vial and irradiated with UV (365 nm) for fluorescence observation (FIG. 28). The fluorescence could be detected only in AuNR @ TEOS-APTES-Dansyl.

### Example 7

### Fluorescence Spectral Measurement and Fluorescence Quantum Yield Calculation for AuNR@TEOS-APTES-Dansyl

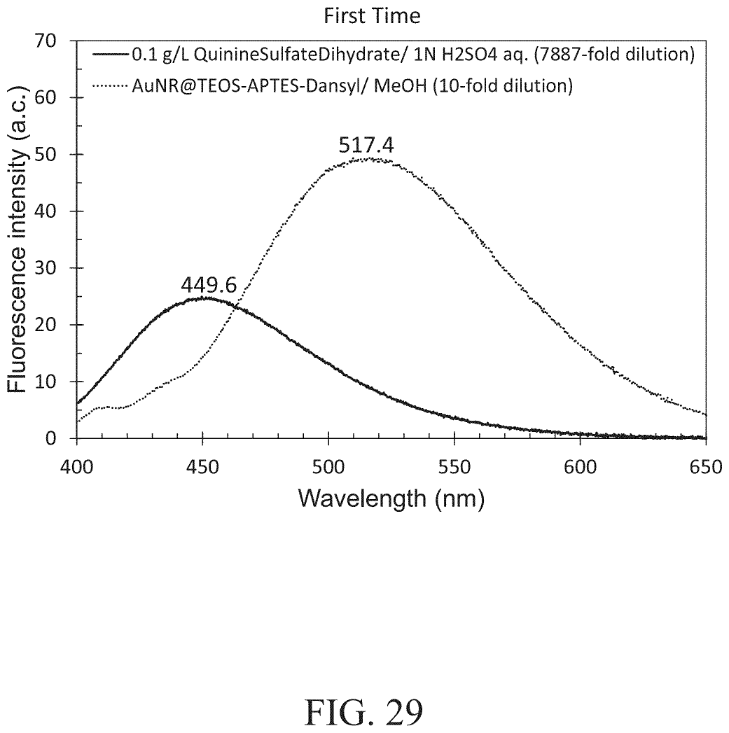

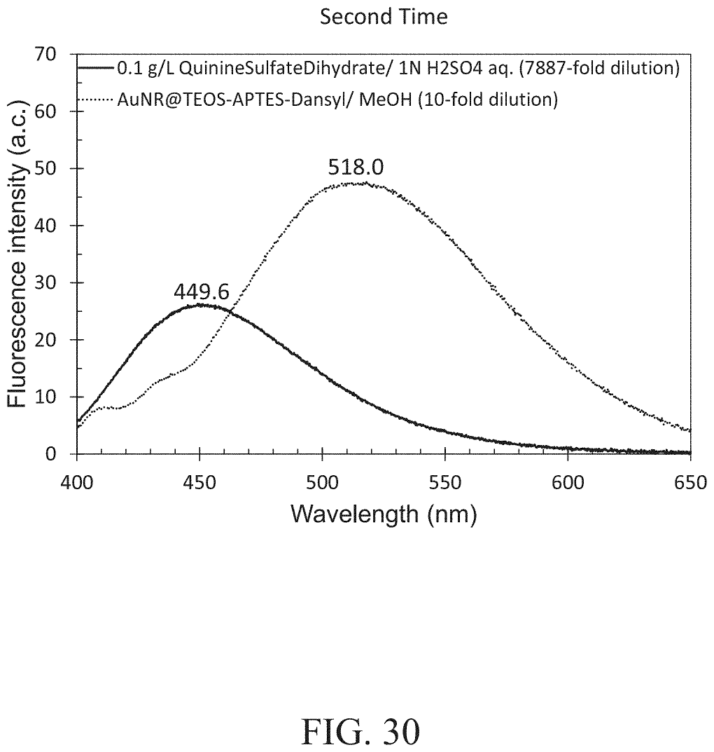

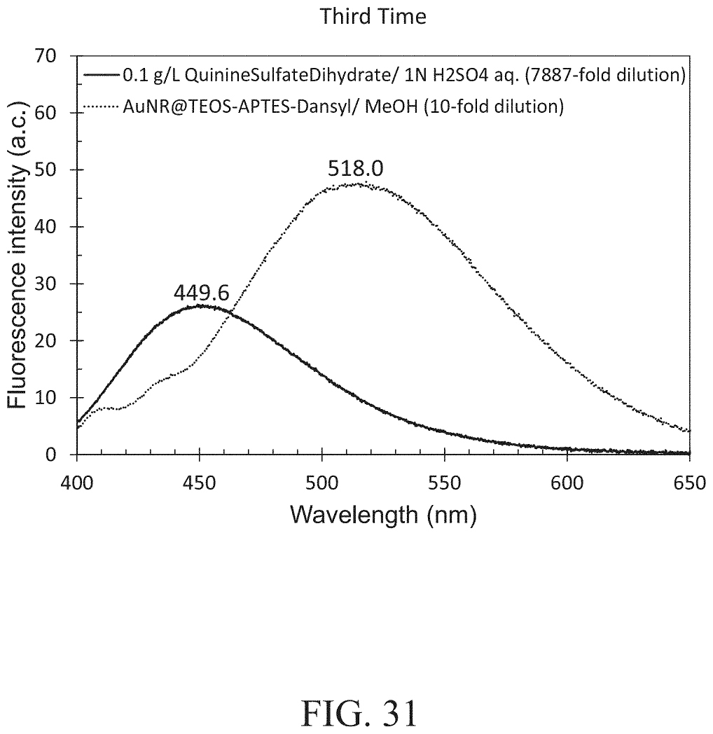

The fluorescence quantum yield of AuNR@TEOS-APTES-Dansyl was calculated by a relative method (FIGS. 29, 30 and 31). The fluorescence quantum yield was calculated using the integrated area of the measured fluorescence spectrum and the absorbance of the absorption spectrum (Ex: 335 nm). The mean and standard deviation of the fluorescence quantum yields were calculated by calculating the fluorescence quantum yield three times in total. The formula for calculating the fluorescence quantum yield using the relative method is shown below.

$${Φ}_{x} = {Φ}_{st} \times ( \frac{{A}_{st}}{{A}_{x}} ) \times ( \frac{{F}_{x}}{{F}_{st}} ) \times ( \frac{{n}_{x}^{2}}{{n}_{st}^{2}} ) \times ( \frac{{D}_{x}}{{D}_{st}} ) \qquad ( 1 )$$

The values measured by the absorption spectrum measurement and the fluorescence spectrum measurement shown in Tables 5 to 7 were substituted into the formula (1) to calculate the relative quantum yield Φ_{F} of AuNR@ TEOS-APTES-Dansyl (standard substance: Quinine Sulfate Dihydrate, unknown sample: AuNR@TEOS-APTES-Dansyl (the spectrum of the difference between it and AuNR@TEOS-APTES was used for the absorbance of the excitation wavelength), Ex: 335.0 nm).

TABLE 5
Fist time

| Item | Sample | Value |
|---|---|---|
| Quantum yield | Standard substance | Φₛₜ = 0.55 |
| Absorbance at excitation | Standard substance | Aₛₜ = 0.20148 |
| wavelength | Unknown sample | Aₓ = 0.04244 |
| Fluorescence spectrum area | Standard substance | Fₛₜ = 1521.704 |
| | Unknown sample | Fₓ = 5322.067 |
| Average refractive index of | Standard substance | nₛₜ =1.3391 |
| solvent | Unknown sample | nₓ = 1.3292 |
| Dilution rate | Standard substance | Dₛₜ = 1000 |
| | Unknown sample | Dₓ = 10 |

${Φ}_{x} = 0.55 \times ( \frac{0.201 \; 48}{0.042 \; 44} ) \times ( \frac{5322.067}{1521.704} ) \times ( \frac{{( 1.329 \; 2 )}^{2}}{{( 1.339 \; 1 )}^{2}} ) \times ( \frac{10}{1000} )$
Φₓ = 0.55 × (4.7474) × (3.4974) × (0.9853) × (0.01)
Φₓ ≈ 0.090

TABLE 6
Second time

| Item | Sample | Value |
|---|---|---|
| Quantum yield | Standard substance | Φₛₜ = 0.55 |
| Absorbance at excitation | Standard substance | Aₛₜ = 0.20148 |
| wavelength | Unknown sample | Aₓ = 0.04244 |
| Fluorescence spectrum area | Standard substance | Fₛₜ = 1697.339 |
| | Unknown sample | Fₓ = 5196.190 |
| Average refractive index of | Standard substance | nₛₜ = 1.3391 |
| solvent | Unknown sample | nₓ = 1.3292 |
| Dilution rate | Standard substance | Dₛₜ = 1000 |
| | Unknown sample | Dₓ = 10 |

${Φ}_{x} = 0.55 \times ( \frac{0.201 \; 48}{0.042 \; 44} ) \times ( \frac{5196.190}{1697.339} ) \times ( \frac{{( 1.329 \; 2 )}^{2}}{{( 1.339 \; 1 )}^{2}} ) \times ( \frac{10}{1000} )$
Φₓ = 0.55 × (4.7474) × (3.0614) × (0.9853) × (0.01)
Φₓ ≈ 0.079

TABLE 7
Third time

| Item | Sample | Value |
|---|---|---|
| Quantum yield | Standard substance | Φₛₜ = 0.55 |
| Absorbance at excitation | Standard substance | Aₛₜ = 0.20148 |
| wavelength | Unknown sample | Aₓ = 0.04244 |
| Fluorescence spectrum area | Standard substance | Fₛₜ = 1774.530 |
| | Unknown sample | Fₓ = 5405.072 |
| Average refractive index of | Standard substance | nₛₜ = 1.3391 |
| solvent | Unknown sample | nₓ = 1.3292 |
| Dilution rate | Standard substance | Dₛₜ = 1000 |
| | Unknown sample | Dₓ = 10 |

${Φ}_{x} = 0.55 \times ( \frac{0.2014 \; 8}{0.042 \; 44} ) \times ( \frac{5405.072}{1774.530} ) \times ( \frac{{( 1.3292 )}^{2}}{{( 1.339 \; 1 )}^{2}} ) \times ( \frac{10}{1000} )$
Φₓ = 0.55 × (4.7474) × (3.0459) × (0.9853) × (0.01)
Φₓ ≈ 0.078

From these three calculations, the fluorescence quantum yield of AuNR@TEOS-APTES-Dansyl was Φ_{F}=8.23+0.67%. This result indicates that AuNR@TEOS-APTES-Dansyl is fluorescent. When the fluorescence spectra of AuNR, AuNR @ TEOS, and AuNR @ TEOS-APTES were measured as control, no fluorescence could be detected from these samples (see FIG. 27). From these results, the fluorescence of AuNR@TEOS-APTES-Dasnyl was thought to be derived from the Dansyl group. From these results, the Dansyl group was successfully modified on the surface of the particles. Furthermore, the fluorescence of AuNR@TEOS-APTES-Dansyl could be detected.

As described above, the present invention achieves a stable and highly sensitive luminescent agent because quenching phenomena due to light energy transfer, etc. near the gold nanorod interface is avoided by silica layer with a thickness of 15 nm or more. Also, in the above examples, the quenching phenomenon can be avoided despite the large thickness distribution range of the silica layer. Therefore, the conditions for the thickness of the silica layer at the time of manufacturing are not strict, which makes it easy to manufacture and reduces the manufacturing cost.

The silica-coated gold nanorods bonded with the labeled materials of the present invention can be used, for example, as contrast agents, nano-therapeutics, bio-imaging agents, and labeling agents. At the time of use, both the emission of the fluorescent agent Dansyl and the diffraction of light by the gold nanorods can be used, making it useful as a bio-imaging agent as it can be observed by both fluorescence and electron microscopy. For example, when administered to a cell or the like in vitro, it is possible to determine which cells have been incorporated with the gold nanorods using a fluorescence microscope, and then make more detailed observations with an electron microscope. Similarly, when administered to laboratory animals, it is possible to determine which organs have been incorporated into the gold nanorods with a fluorescence microscope, and then make more detailed observations with an electron microscope.

As for the application as the nano-therapeutic agent in the human body, the gold nanorods of the present invention are harmless because gold nanorods are harmless to the human body. First, the silica-coated gold nanorods bonded with the labeling materials of the present invention are delivered to pathological sites such as cancer by a drug delivery system. Second, by irradiating fluorescence as a target with near-infrared radiation, the gold nanorods can be heated up for treatment.

In addition, the present invention that fluoresces a contrast agent, a labeling agent, or the like is useful.

### INDUSTRIAL AVAILABILITY

The silica-coated gold nanorods bonded with the labeling materials in accordance with the present invention can be used, for example, in contrast agents, nano-therapeutics, bio-imaging agents, labeling agents, etc.

We claim:

1. A labeled silica-coated gold nanorod comprising a gold nanorod, a silica layer covering the gold nanorod, spacers bonded to the silica layer, and labeled materials,
wherein the labeled material is chemically bonded to the spacer.

2. The labeled silica-coated gold nanorods according to claim 1, wherein a thickness of the silica layer is 15 nm or more.

3. The labeled silica-coated gold nanorod according to claim 1, wherein the spacer is derived from a silane coupling agent having a Si atom and four functional groups directly or indirectly connected to the Si atom,
the four functional groups have at least one inorganic functional group and at least one organic functional group.

4. The labeled silica-coated gold nanorod according to claim 3, wherein the organic functional group is at least one selected from the group consisting of a vinyl group, an epoxy group, a styryl group, a methacrylic group, an acrylic group, an amino group, an ureide group, an isocyanate group, an isocyanurate group, and a mercapto group.

5. The labeled silica-coated gold nanorod according to claim 3, wherein the organic functional group is indirectly connected to the Si atom via an alkyl group having 1 to 5 carbons, an alkoxy group having 1 to 5 carbons, a phenyl group, a heterocyclic group, or a fused ring group.

6. The labeled silica-coated gold nanorod according to claim 1, wherein the spacer is vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, P-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethylbutylidene)propylamine, N-phenyl-3-aminopropyl trimethoxysilane, 3-ureidopropyltrialkoxysilane, 3-isocyanatepropyltriethoxysilane, tris-[(trimethoxysilyl)propyl]isocyanurate, (3-mercaptopropyl)methyldimethoxysilane, or 3-mercaptopropyltrimethoxysilane.

7. A method for producing a labeled silica-coated gold nanorod, comprising an introduction step and a binding step, wherein
in the introduction step, spacers are introduced on a silica layer of a silica-coated gold nanorod and
in the binding step, a labeled material is chemically bound to the spacer.

8. The method for producing the labeled silica-coated gold nanorods according to claim 7, wherein the thickness of the silica layer is 15 nm or more.
